(19) United States
(12) Patent Application Publication
(10) Pub. No.: US 2005/0187171 A1
(43) Pub. Date: Aug. 25, 2005

(54) Glycomimetic antagonists for both E-and P-selectins

(75) Inventors: John L. Magnani, Gaithersburg, MD (US); John T. Patton JR., Gaithersburg, MD (US); Arun K. Sarkar, North Potomac, MD (US)

Correspondence Address: SEED INTELLECTUAL PROPERTY LAW GROUP PLLC, 701 FIFTH AVE, SUITE 6300, SEATTLE, WA, 98104-7092, US

(73) Assignee: GlycoMimetics, Inc., Gaithersburg, MD (US)

(21) Appl. No.: 10/992,238

(22) Filed: Nov. 18, 2004

Related U.S. Application Data
(60) Provisional application No. 60/523,215, filed on Nov. 19, 2003.
(60) Provisional application No. 60/582,734, filed on Jun. 24, 2004.

(51) Int. Cl. A61K 31/726

(52) U.S. Cl. 514/43; 536/53

(57) ABSTRACT

Compounds and methods are provided for modulating in vitro and in vivo processes mediated by selectin binding. More specifically, selectin modulators and their use are described, wherein the selectin modulators that modulate (e.g., inhibit or enhance) a selectin-mediated function comprise particular glycomimetics linked to a member of a class of compounds termed BASAs (Benzyl Amino Sulfonic Acids).

### CROSS REFERENCE TO RELATED APPLICATIONS

This application claims the benefit of U.S. Provisional Patent Application No. 60/523,215 filed Nov. 19, 2003 and U.S. Provisional Patent Application No. 60/582,734 filed Jun. 24, 2004; where these two provisional applications are incorporated herein by reference in their entireties.

### BACKGROUND OF THE INVENTION

1. Field of the Invention

The present invention relates generally to compounds, compositions and methods for modulating processes mediated by selectin binding, and more particularly to selectin modulators and their use, wherein the selectin modulators that modulate a selectin-mediated function comprise particular glycomimetics linked to a member of a class of compounds termed BASAs (Benzyl Amino Sulfonic Acids, which include a portion or analogue thereof).

2. Description of the Related Art

When a tissue is infected or damaged, the inflammatory process directs leukocytes and other immune system components to the site of infection or injury. Within this process, leukocytes play an important role in the engulfment and digestion of microorganisms. Thus, the recruitment of leukocytes to infected or damaged tissue is critical for mounting an effective immune defense.

Selectins are a group of structurally similar cell surface receptors that are important for mediating leukocyte binding to endothelial cells. These proteins are type 1 membrane proteins and are composed of an amino terminal lectin domain, an epidermal growth factor (EGF)-like domain, a variable number of complement receptor related repeats, a hydrophobic domain spanning region and a cytoplasmic domain. The binding interactions appear to be mediated by contact of the lectin domain of the selectins and various carbohydrate ligands.

There are three known selecting: E-selectin, P-selectin and L-selectin. E-selectin is found on the surface of activated endothelial cells, which line the interior wall of capillaries. E-selectin binds to the carbohydrate sialyl-Lewis^{x} (SLe^{x}), which is presented as a glycoprotein or glycolipid on the surface of certain leukocytes (monocytes and neutrophils) and helps these cells adhere to capillary walls in areas where surrounding tissue is infected or damaged; and E-selectin also binds to sialyl-Lewis^{a} (SLe^{a}), which is expressed on many tumor cells. P-selectin is expressed on inflamed endothelium and platelets, and also recognizes SLe^{x} and SLe^{a}, but also contains a second site that interacts with sulfated tyrosine. The expression of E-selectin and P-selectin is generally increased when the tissue adjacent to a capillary is infected or damaged. L-selectin is expressed on leukocytes. Selectin-mediated intercellular adhesion is an example of a selectin-mediated function.

Modulators of selectin-mediated function include the PSGL-1 protein (and smaller peptide fragments), fucoidan, glycyrrhizin (and derivatives), anti-selectin antibodies, sulfated lactose derivatives, and heparin. All have shown to be unsuitable for drug development due to insufficient activity, toxicity, lack of specificity, poor ADME characteristics and/or availability of material.

Although selectin-mediated cell adhesion is required for fighting infection and destroying foreign material, there are situations in which such cell adhesion is undesirable or excessive, resulting in tissue damage instead of repair. For example, many pathologies (such as autoimmune and inflammatory diseases, shock and reperfusion injuries) involve abnormal adhesion of white blood cells. Such abnormal cell adhesion may also play a role in transplant and graft rejection. In addition, some circulating cancer cells appear to take advantage of the inflammatory mechanism to bind to activated endothelium. In such circumstances, modulation of selectin-mediated intercellular adhesion may be desirable.

Accordingly, there is a need in the art for identifying inhibitors of selectin-mediated function, e.g., of selectin-dependent cell adhesion, and for the development of methods employing such compounds to inhibit conditions associated with excessive selectin activity. The present invention fulfills these needs and further provides other related advantages.

### BRIEF SUMMARY OF THE INVENTION

Briefly stated, this invention provides compounds, compositions and methods for modulating selectin-mediated processes. In the present invention, the compounds that modulate (e.g., inhibit or enhance) a selectin-mediated function contain a particular glycomimetic and a BASA (i.e., a benzyl amino sulfonic acid or portion or analogue of either). Such compounds may be combined with a pharmaceutically acceptable carrier or diluent to form a pharmaceutical composition. The compounds or compositions may be used in a method to modulate (e.g., inhibit or enhance) a selectin-mediated function, such as inhibiting a selectin-mediated intercellular adhesion.

In one aspect of the present invention, compounds are provided that contain at least two components: (1) a particular glycomimetic (or glycoconjugate thereof) and (2) a BASA. Examples of a BASA are set forth below. Preferred are the BASAs shown in FIGS. 1A-1I. Examples of preferred glycomimetics are shown in FIG. 1J. A compound of the present invention is a combination of a particular glycomimetic and a BASA, to yield a compound that modulates (e.g., inhibits or enhances) a selectin-mediated function. A BASA may be attached at R, R′ or R″ of FIG. 1J and replace the substituent at that position. An example of a selectin-mediated function is a selectin-mediated intercellular adhesion. A compound of the present invention includes physiologically acceptable salts thereof. A compound of the present invention in combination with a pharmaceutically acceptable carrier or diluent provides a composition of the present invention.

In the preferred embodiments of the present invention, a compound or physiologically acceptable salt thereof is provided having the formula: 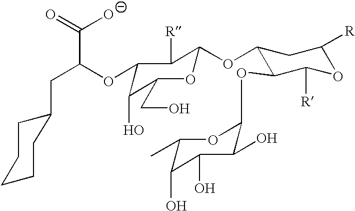
wherein:
R=H or a benzyl amino sulfonic acid;
R′=a benzyl amino sulfonic acid, 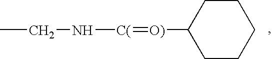 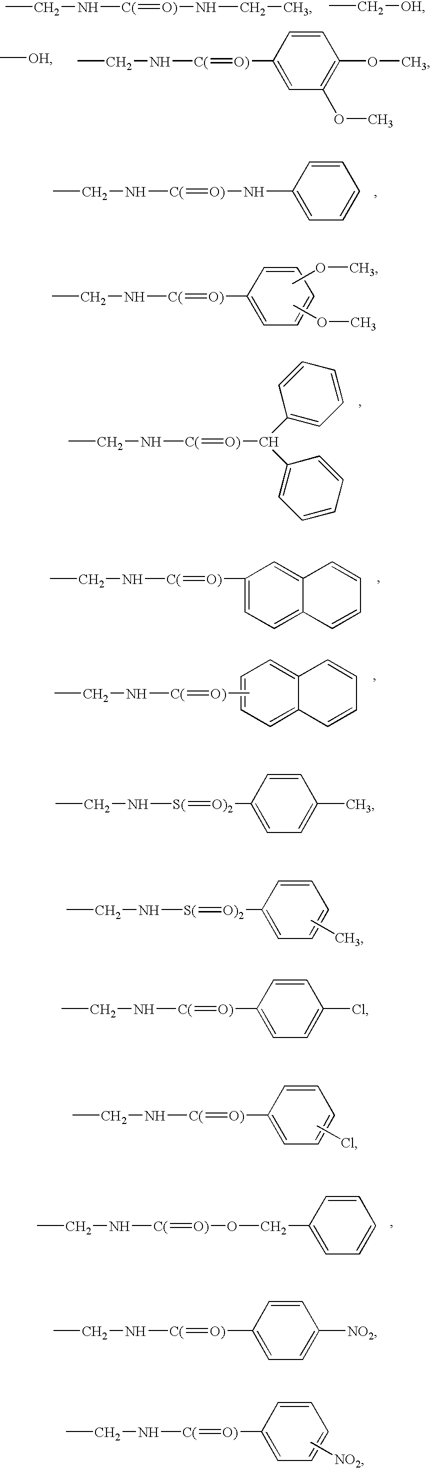 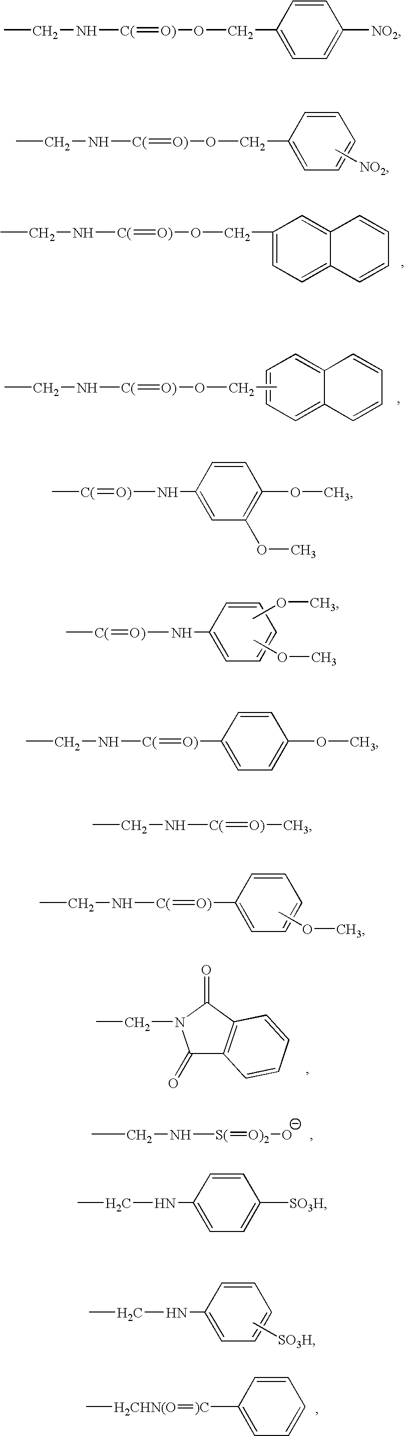 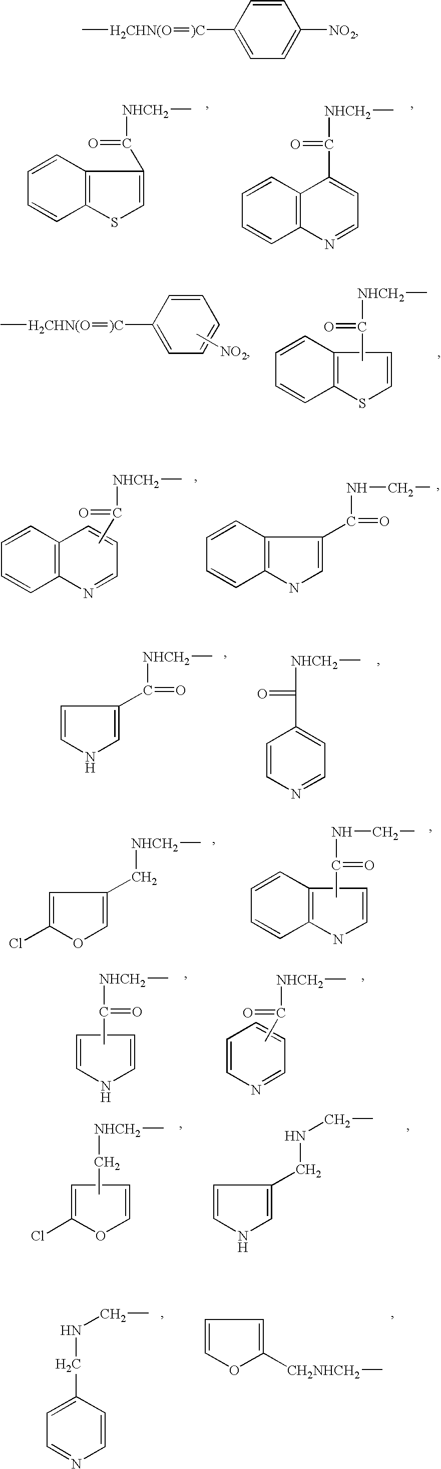 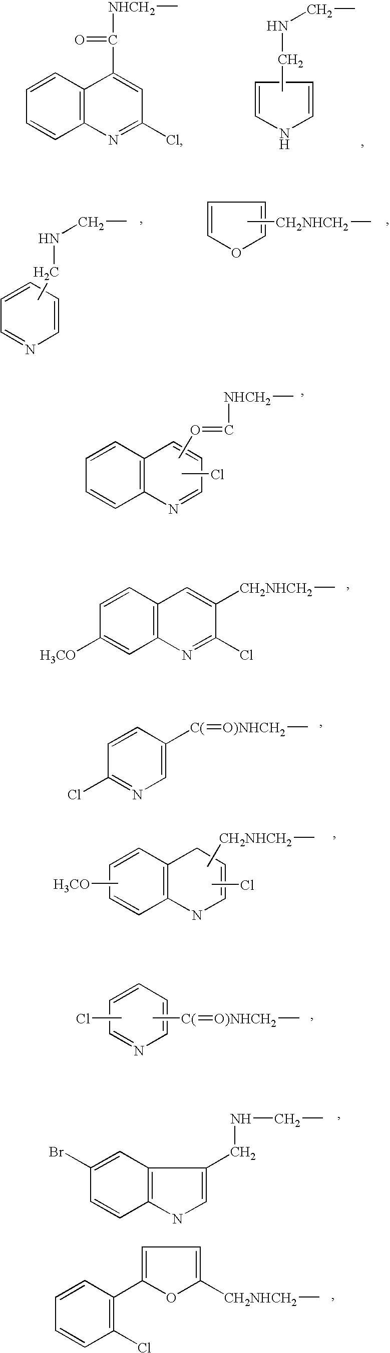 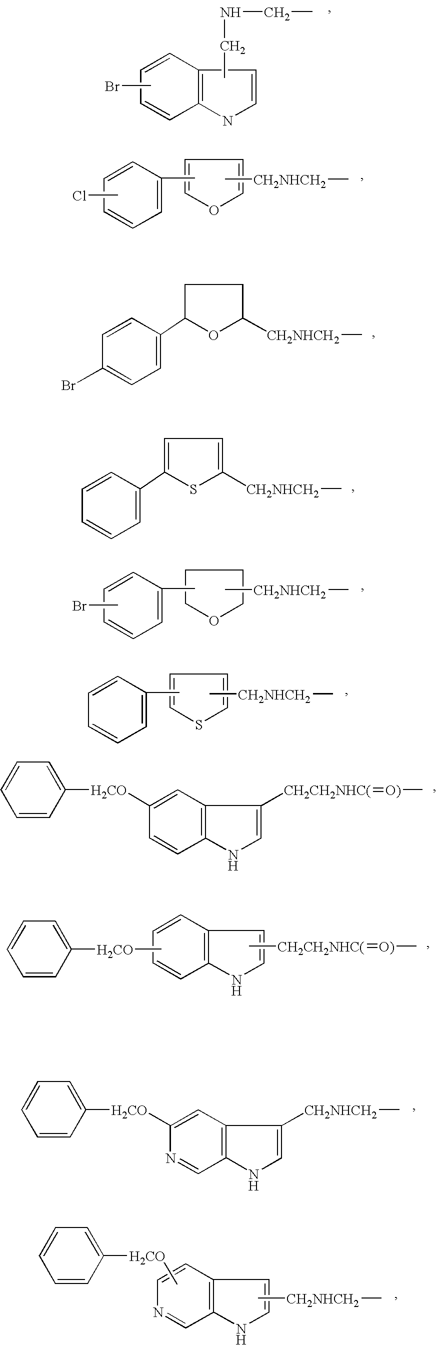 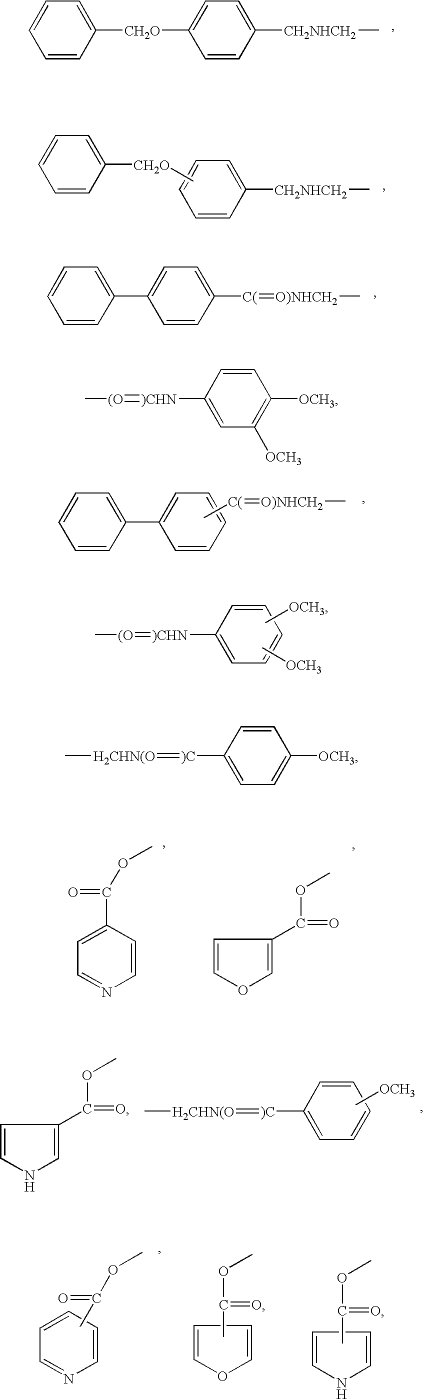 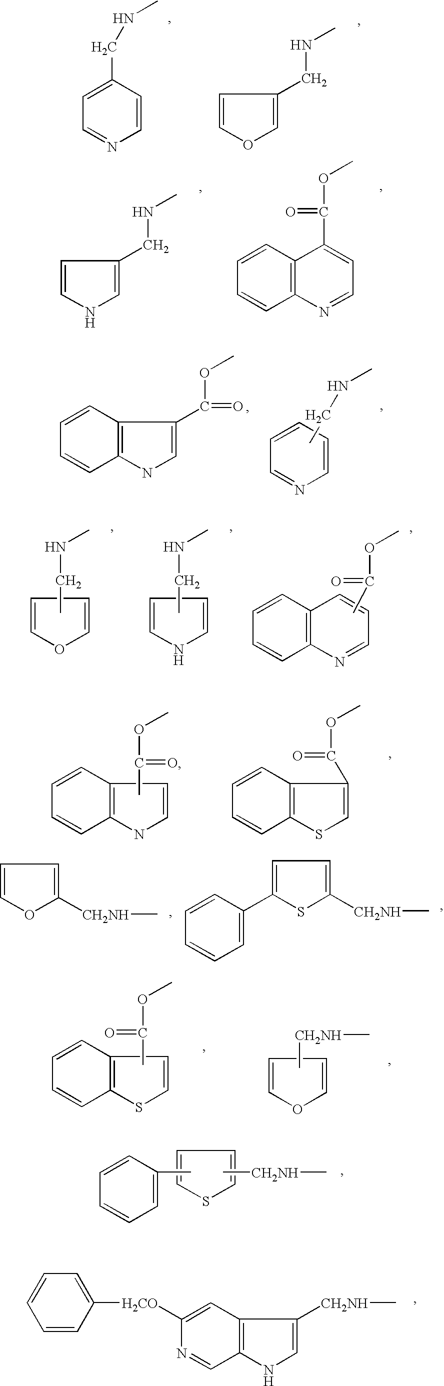 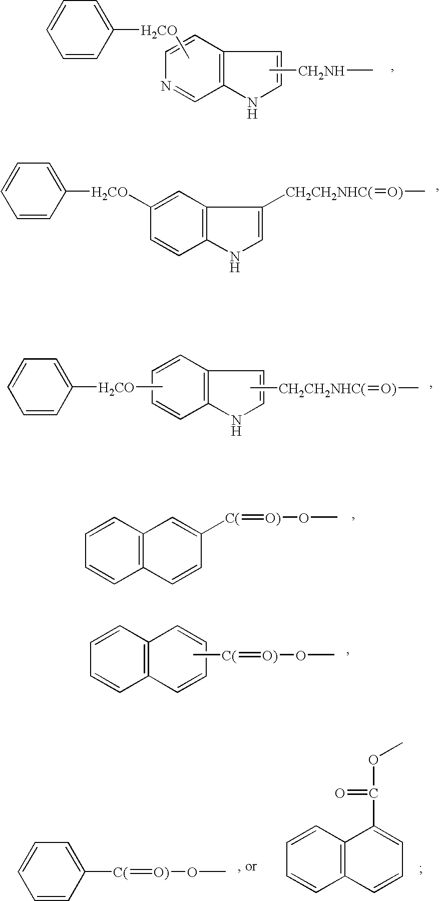
R″=a benzyl amino sulfonic acid, —OH, —OC(═O)—NH—CH₂—CH₃, 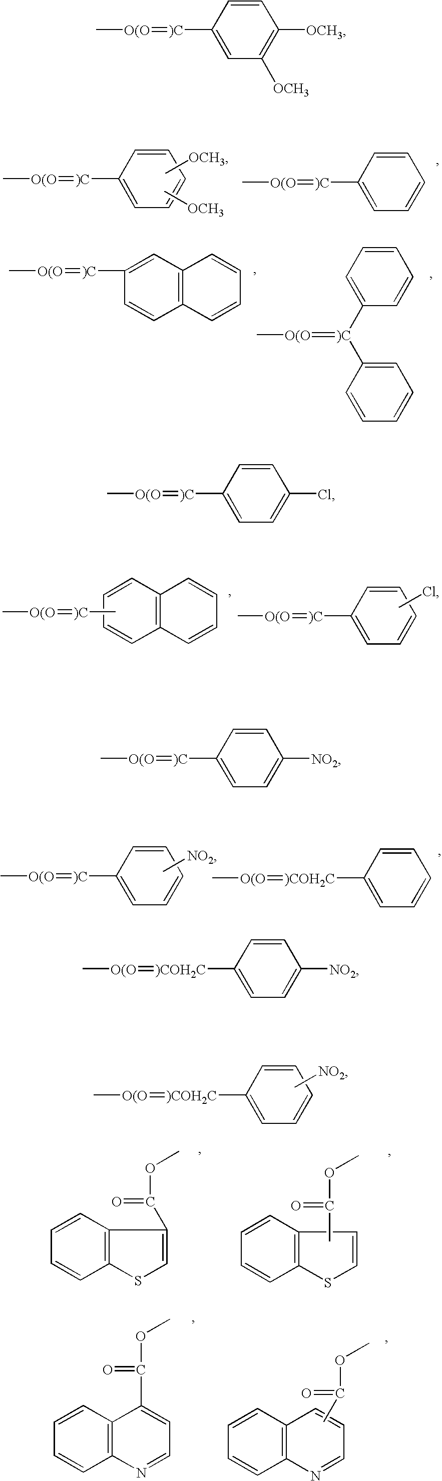 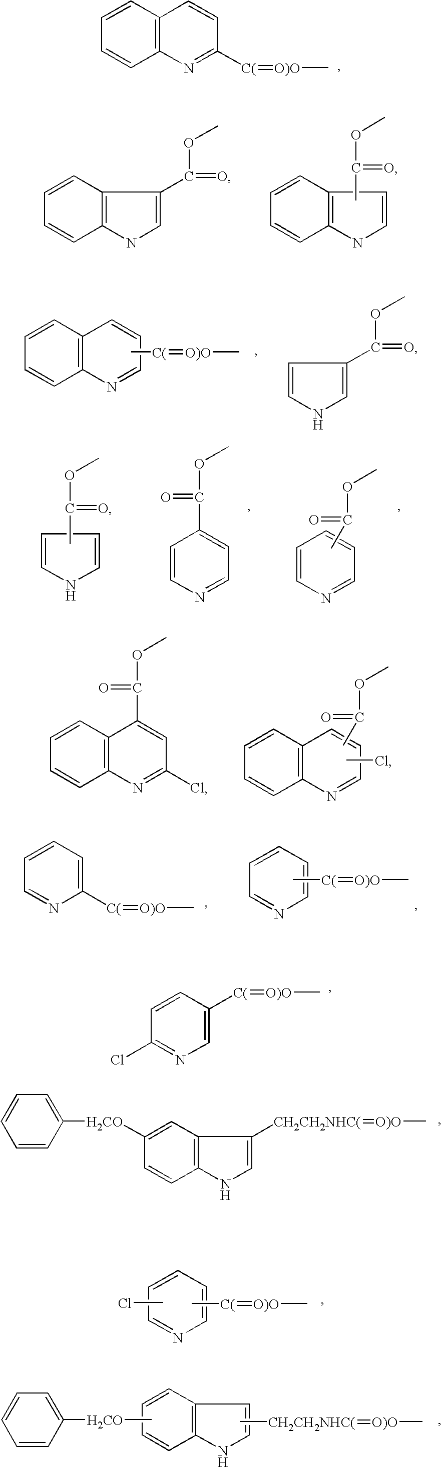 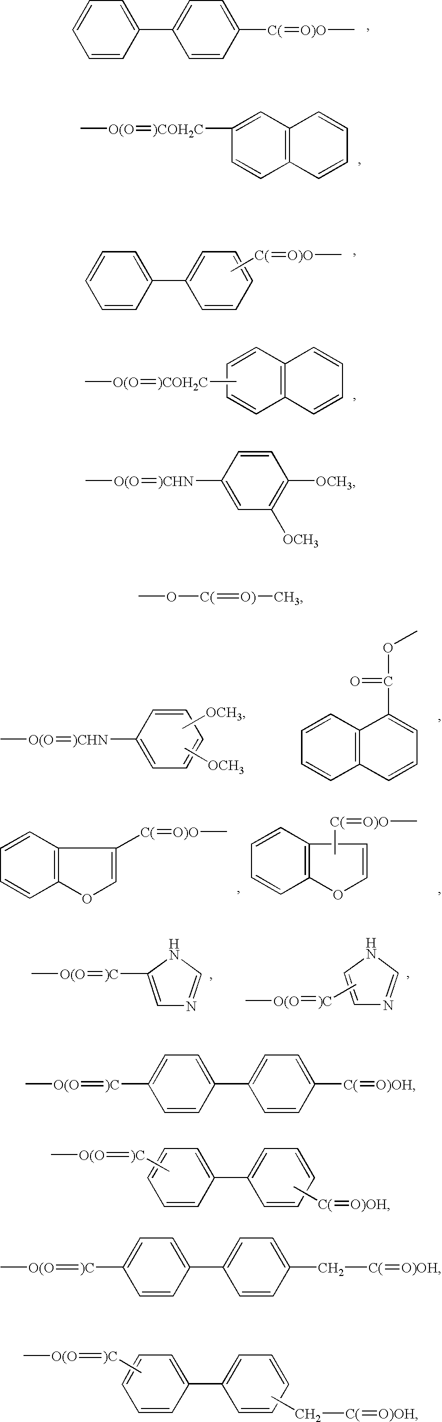 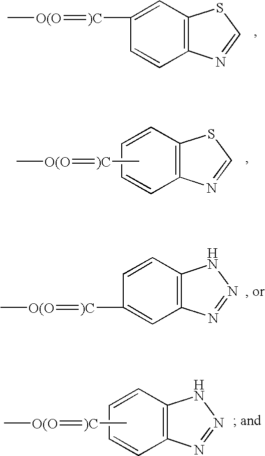
   wherein the compound possesses a benzyl amino sulfonic acid at R, R′ or R″ but not at more than one of R, R′ and R″. Such a compound may be combined with a pharmaceutically acceptable carrier or diluent to provide a preferred composition of the present invention. A compound or composition of the present invention may further comprise a diagnostic or therapeutic agent. In the chemical formulae herein (including the figures), a line through the middle of another line represents attachment of the substituent at any one of the carbon atoms within a ring (or rings if fused). The individual compounds formed by selection of a particular substituent for each of R, R′ and R″ from the substituents set forth above are all disclosed by the present application, by the listing of the substituents, to the same extent as if each and every combination of substituents for R, R′ and R″ were separately listed.

In another aspect of the present invention, methods are provided for using a compound or composition of the present invention to modulate a selectin-mediated function. Such a compound or composition can be used, for example, to inhibit or enhance a selectin-mediated function, such as selectin-mediated intercellular interactions. A compound or composition can be used in a method to contact a cell expressing a selectin in an amount effective to modulate the selectin's function. A compound or composition can be used in a method to administer to a patient, who is in need of having inhibited the development of a condition associated with an excessive selectin-mediated function (such as an excessive selectin-mediated intercellular adhesion), in an amount effective to inhibit the development of such a condition. Examples of such conditions include inflammatory diseases, autoimmune diseases, infection, cancer, shock, thrombosis, wounds, burns, reperfusion injury, platelet-mediated diseases, leukocyte-mediated lung injury, spinal cord damage, digestive tract mucous membrane disorders, osteoporosis, arthritis, asthma and allergic reactions. A compound or composition can be used in a method to administer to a patient who is the recipient of a transplanted tissue in an amount effective to inhibit rejection of the transplanted tissue. A compound or composition can be used in a method in an amount effective to target an agent (e.g., a diagnostic or therapeutic agent) to a selectin-expressing cell by contacting such a cell with the agent linked to the compound or composition. A compound or composition can be used in the manufacture of a medicament, for example for any of the uses recited above.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE SEVERAL VIEWS OF THE DRAWINGS

FIGS. 1A-1I show structures of representative BASA components of the selectin modulators as described herein. The compounds illustrated in these figures include BASA portions and analogues. FIG. 1J shows structures of preferred glycomimetic components of the selectin modulators as described herein.

FIG. 2 is a diagram illustrating the synthesis of a representative BASA.

FIG. 3 is a diagram illustrating the synthesis of a representative BASA.

FIG. 4 is a diagram illustrating the synthesis of a glycomimetic.

FIG. 5 is a diagram illustrating the synthesis of a glycomimetic.

FIG. 6A is a diagram illustrating the synthesis of a glycomimetic precursor.

FIG. 6B is a diagram illustrating the synthesis of several glycomimetics via use of the precursor of FIG. 6A.

FIGS. 7A and 7B are diagrams illustrating the synthesis of glycomimetic-BASA compounds.

FIG. 8A is a diagram illustrating the synthesis of a glycomimetic precursor.

FIG. 8B is a diagram illustrating the synthesis of several glycomimetics via use of the precursor of FIG. 8A.

FIG. 9A is a diagram illustrating the synthesis of a glycomimetic precursor.

FIG. 9B is a diagram illustrating the synthesis of several glycomimetics via use of the precursor of FIG. 9A.

FIG. 10 is a diagram illustrating the synthesis of a glycomimetic-BASA compound.

FIG. 11 is a diagram illustrating the synthesis of a glycomimetic-BASA compound.

FIG. 12 is a diagram illustrating the syntheses of a BASA and a BASA-squarate.

FIG. 13 is a diagram illustrating the synthesis of a glycomimetic-BASA compound.

FIG. 14 is a diagram illustrating the synthesis of a glycomimetic-BASA compound.

FIGS. 15A and 15B are diagrams illustrating the syntheses of glycomimetic-BASA compounds.

FIGS. 16A and 16B are diagrams illustrating the syntheses of glycomimetic-BASA compounds.

### DETAILED DESCRIPTION OF THE INVENTION

As noted above, the present invention provides selectin modulators, compositions thereof and methods for modulating selectin-mediated functions. Such modulators may be used in vitro or in vivo, to modulate (e.g., inhibit or enhance) selectin-mediated functions in a variety of contexts, discussed in further detail below. Examples of selectin-mediated functions include intercellular adhesion and the formation of new capillaries during angiogenesis.

Selectin Modulators

The term “selectin modulator,” as used herein, refers to a molecule(s) that modulates (e.g., inhibits or enhances) a selectin-mediated function, such as selectin-mediated intercellular interactions, and that comprises at least one of the following BASA:
(a) a BASA (or a salt thereof);
(b) a portion of a BASA that retains the ability to modulate (e.g., inhibit or enhance) a selectin-mediated function; or
(c) an analogue of a BASA, or an analogue of a portion of a BASA, that has the ability to modulate (e.g., inhibit or enhance) a selectin-mediated function;
   wherein at least one of (a), (b) or (c) is linked to one or more particular selectin-binding glycomimetic (or glycoconjugate thereof).

A selectin modulator may consist entirely of one or more of the above BASA elements linked to one or more particular glycomimetic, or may comprise one or more additional molecular components. The selectin modulators of the present invention are, surprisingly, significantly more potent than the individual components alone or additively.

Within the present invention, BASAs are low molecular weight sulfated compounds which have the ability to interact with a selectin. The interaction modulates or assists in the modulation (e.g., inhibition or enhancement) of a selectin-mediated function (e.g., an intercellular interaction). They exist as either their protonated acid form, or as a sodium salt, although sodium may be replaced with potassium or any other pharmaceutically acceptable counterion. A representative BASA has the following structure:

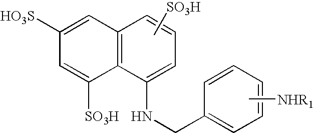

Portions of BASA that retain the ability to interact with a selectin (which interaction modulates or assists in the modulation of a selectin-mediated function as described herein) are also a BASA component of the selectin modulators of the present invention. Such portions generally comprise at least one aromatic ring present within the BASA structure. Within certain embodiments, a portion may comprise a single aromatic ring, multiple such rings or half of a symmetrical BASA molecule.

As noted above, analogues of BASA and portions thereof (both of which possess the biological characteristic set forth above) are also encompassed, e.g., by the BASA component of the selectin modulators, within the present invention. As used herein, an “analogue” is a compound that differs from BASA or a portion thereof because of one or more additions, deletions and/or substitutions of chemical moieties, such that the ability of the analogue to inhibit a selectin-mediated interaction is not diminished. For example, an analogue may contain S to P substitutions (e.g., a sulfate group replaced with a phosphate group). Other possible modifications include: (a) modifications to ring size (e.g., any ring may contain between 4 and 7 carbon atoms); (b) variations in the number of fused rings (e.g., a single ring may be replaced with a polycyclic moiety containing up to three fused rings, a polycyclic moiety may be replaced with a single unfused ring or the number of fused rings within a polycyclic moiety may be altered); (c) ring substitutions in which hydrogen atoms or other moieties covalently bonded to a carbon atom within an aromatic ring may be replaced with any of a variety of moieties, such as F, Cl, Br, I, OH, O-alkyl (C1-8), SH, NO₂, CN, NH₂, NH-alkyl (C1-8), N-(alkyl)₂, SO₃M (where M=H⁺, Na⁺, K⁺ or other pharmaceutically acceptable counterion), CO₂M, PO₄M₂, SO₂NH₂, alkyl (C1-8), aryl (C6-10), CO₂-alkyl (C1-8), —CF₂X (where X can be H, F, alkyl, aryl or acyl groups) and carbohydrates; and (d) modifications to linking moieties (i.e., moieties located between rings in the BASA molecule) in which groups such as alkyl, ester, amide, anhydride and carbamate groups may be substituted for one another.

Certain BASA portions and analogues contain one of the following generic structures: 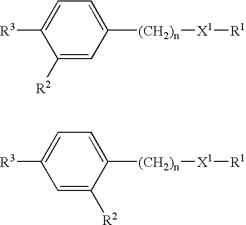
Within this structure, n may be 0 or 1, X¹ may be —PO₂M, —SO₂M or —CF₂— (where M is a pharmaceutically acceptable counterion such as hydrogen, sodium or potassium), R¹ may be —OH, —F or —CO₂R⁴ (where R⁴ may be —H or —(CH₂)ₘ—CH₃ and m is a number ranging from 0 to 3, R² may be —H, —PO₃M₂, —SO₃M₂, —CH₂—PO₃M₂, —CH₂—SO₃M₂, —CF₃ or —(CH₂)ₘ—C(R⁶)H—R⁵ or R⁹—N(R¹⁰)—, R³ may be —H, —(CH₂)ₘ—C(R⁶)H—R⁵ or R⁹—N(R¹⁰)— (where R⁵ and R⁶ may be independently selected from —H, —CO₂—R⁷ and —NH—R⁸, R⁷ and R⁸ may be independently selected from hydrogen and moieties comprising one or more of an alkyl group, an aromatic moiety, an amino group or a carboxy group, and R⁹ and R¹⁰ may be independently selected from —H, —(CH₂)ₘ—CH₃; —CH₂—Ar, —CO—Ar, where m is a number ranging from 0 to 3 and Ar is an aromatic moiety (i.e., any moiety that comprises at least one substituted or unsubstituted aromatic ring, wherein the ring is directly bonded to the —CH₂— or —CO— group indicated above)).

Other portions and analogues of BASA comprise the generic structure: 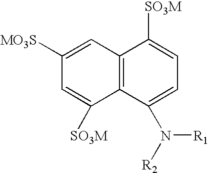
Within this structure, R₁ and R₂ may be independently selected from (i) hydrogen, (ii) moieties comprising one or more of an alkyl group, an aromatic moiety, an amino group or a carboxy group, and (iii) —CO—R₃ (where R₃ comprises an alkyl or aromatic moiety as described above) and M is a pharmaceutically acceptable counterion.

The individual compounds, or groups of compounds, derived from the various combinations of the structures and substituents described herein, are disclosed by the present application to the same extent as if each compound or group of compounds was set forth individually. Thus, selection of particular structures and/or particular substituents is within the scope of the present invention.

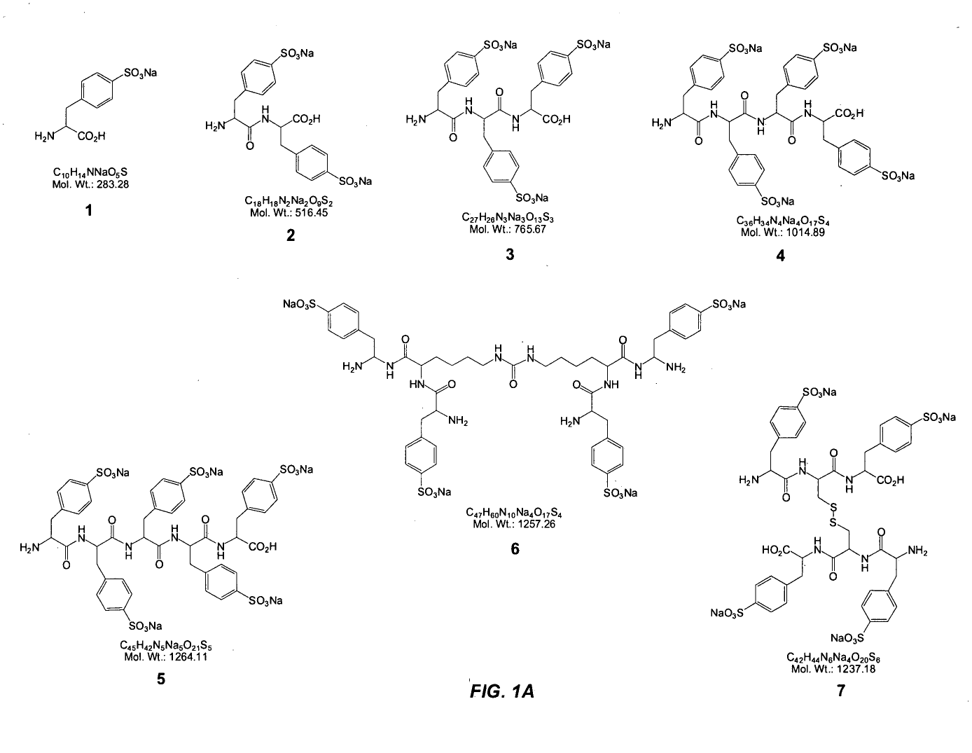

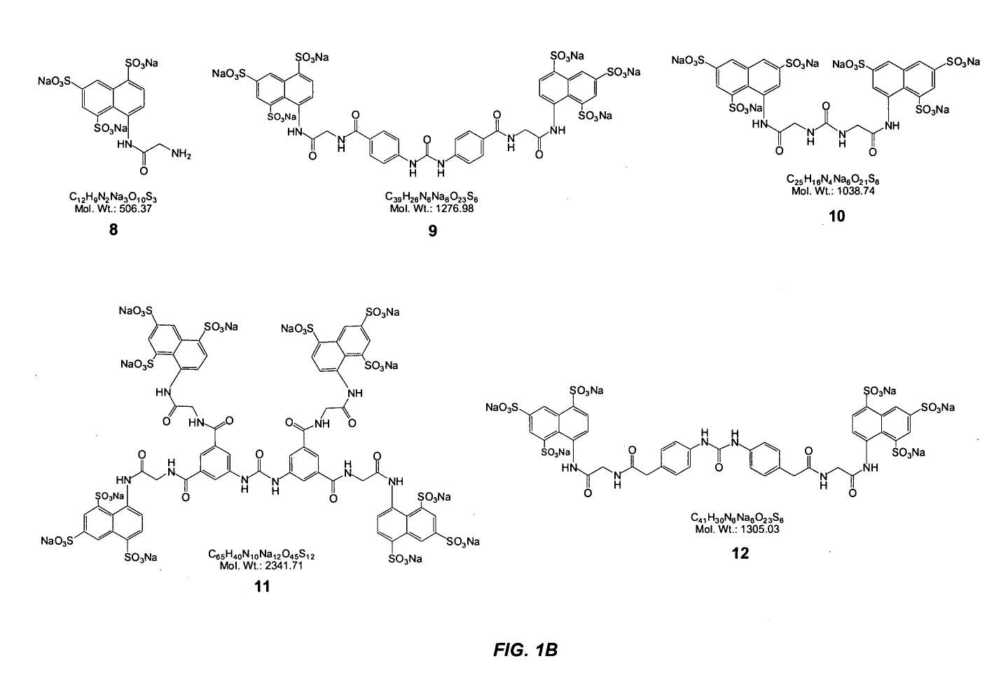

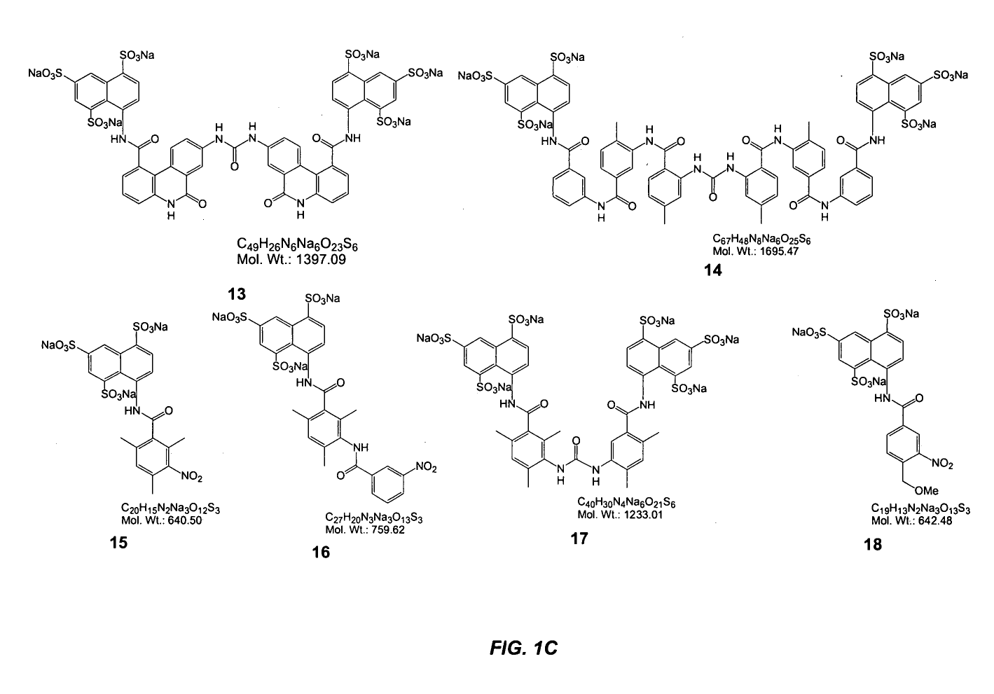

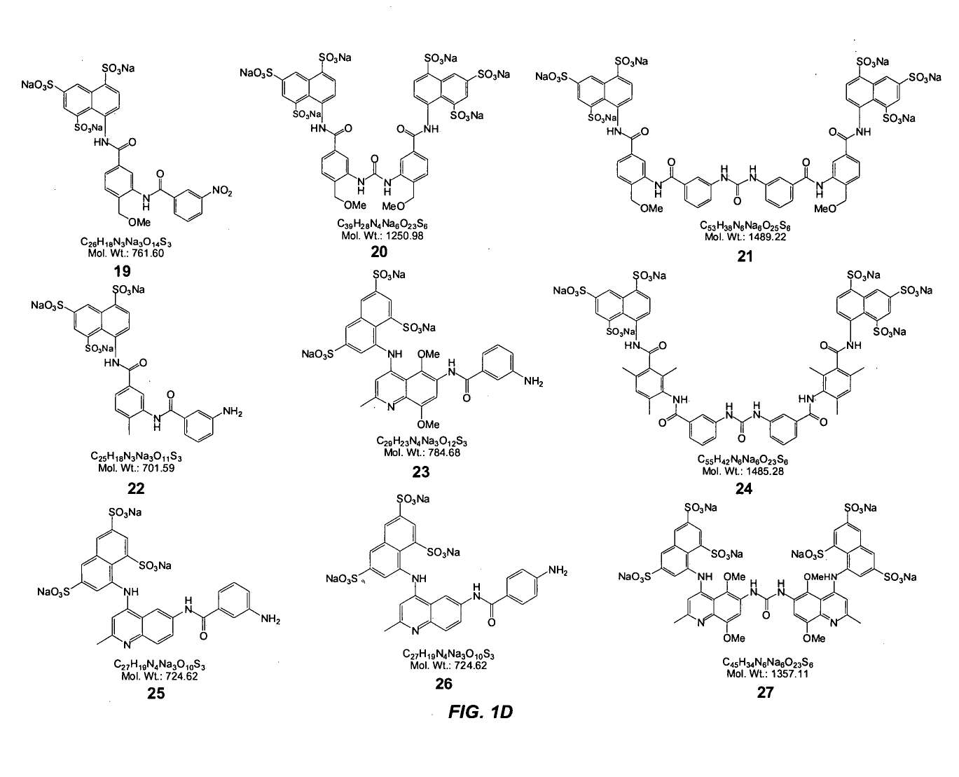

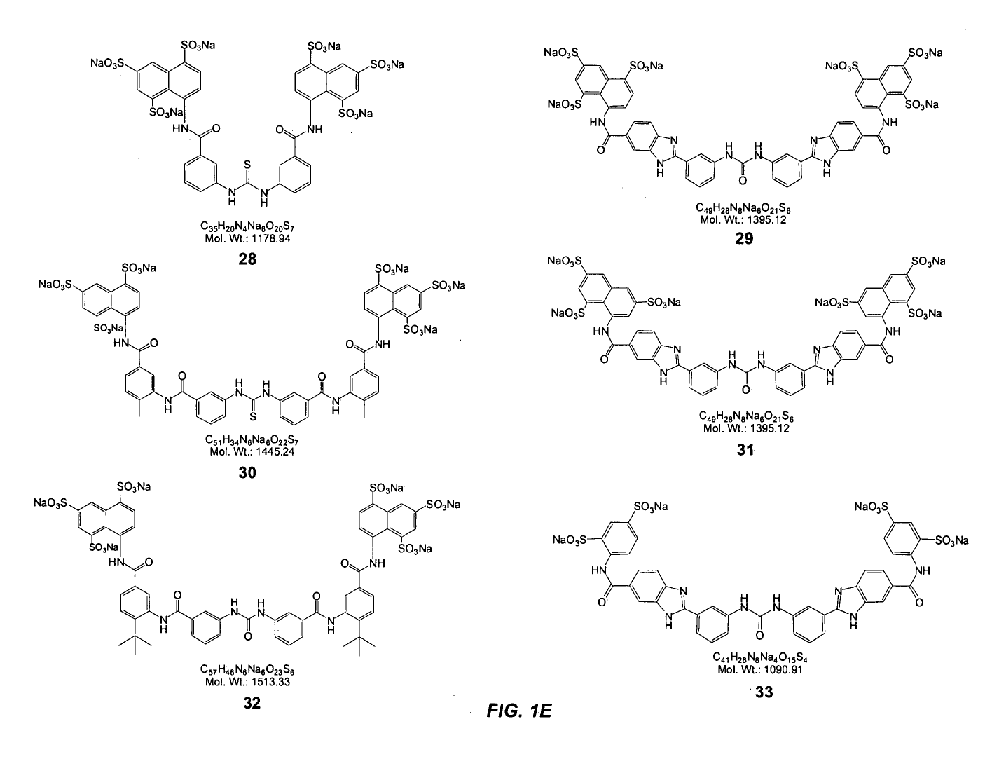

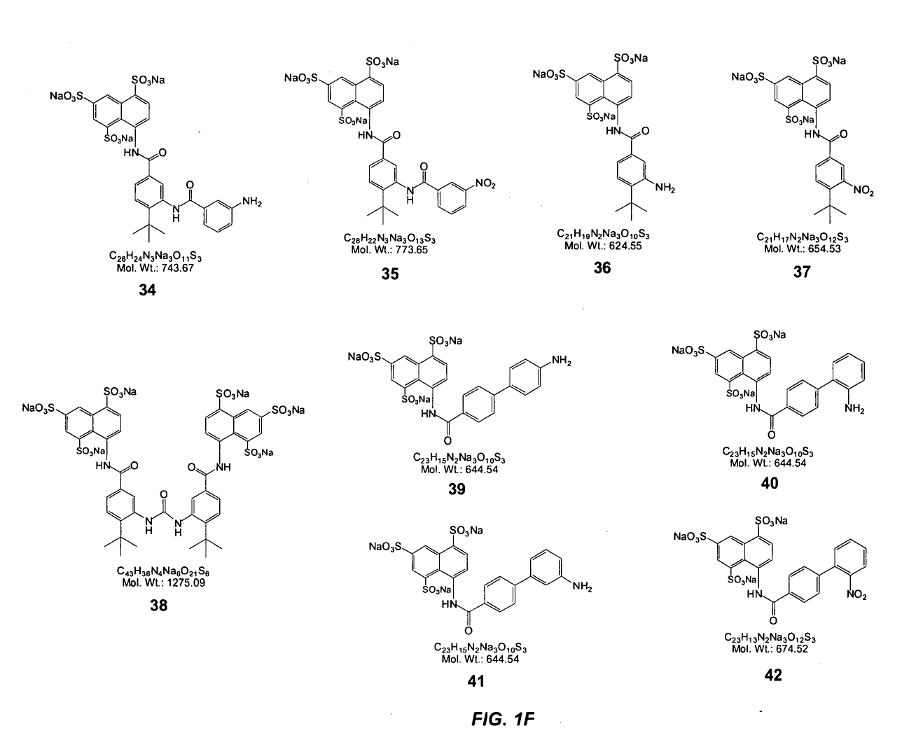

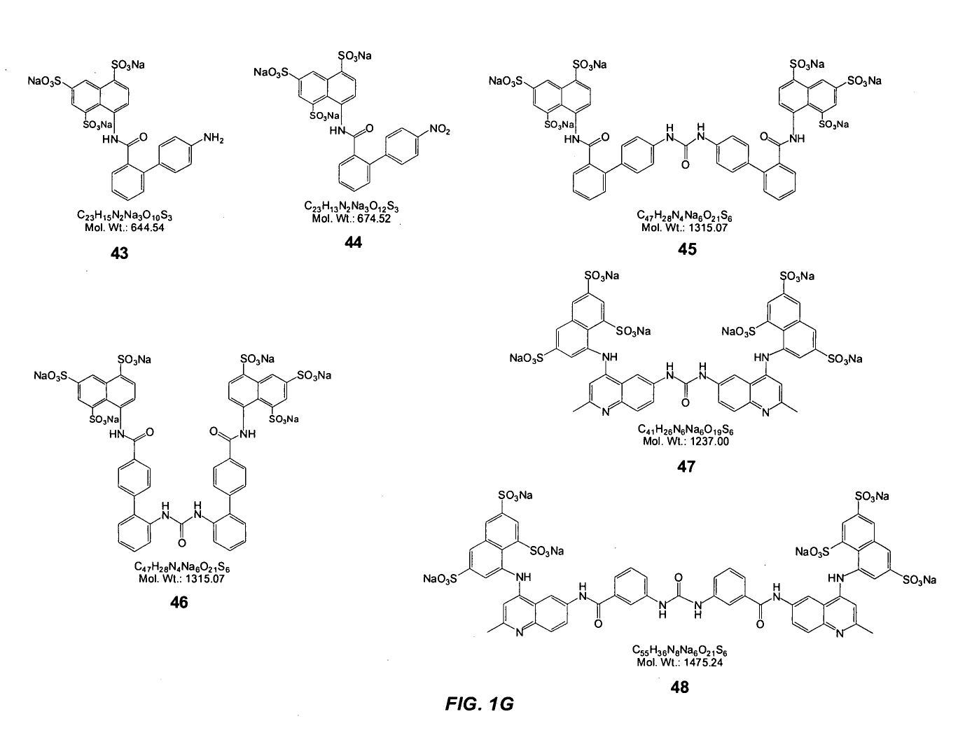

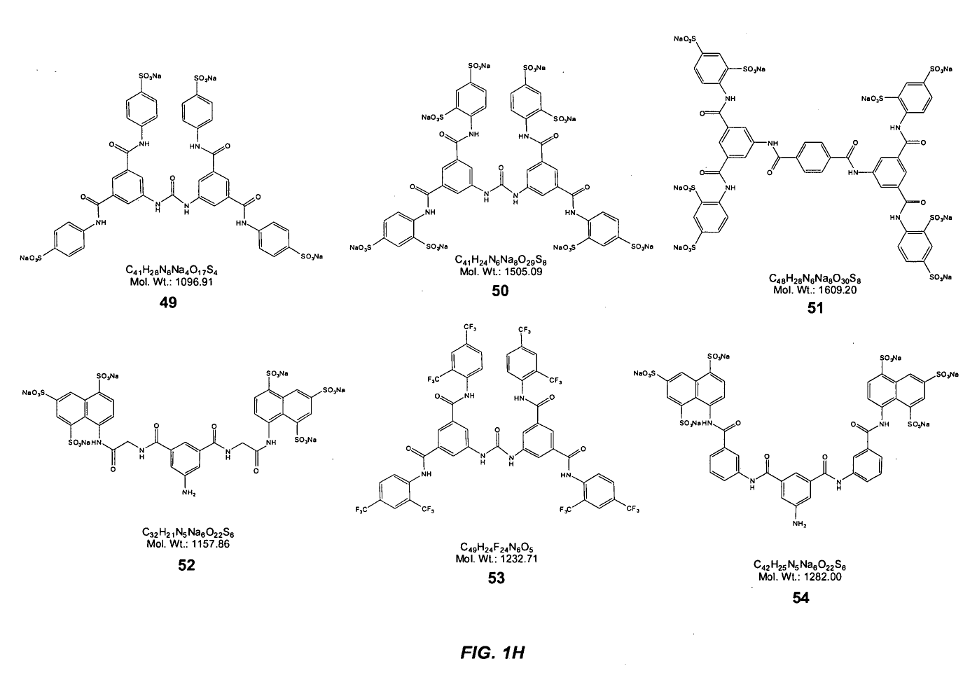

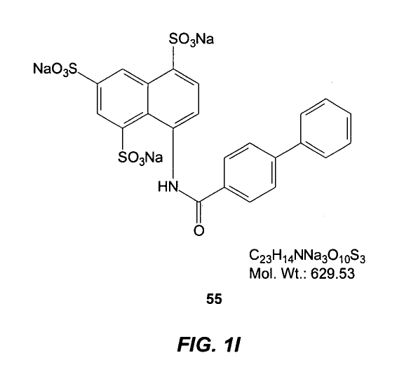

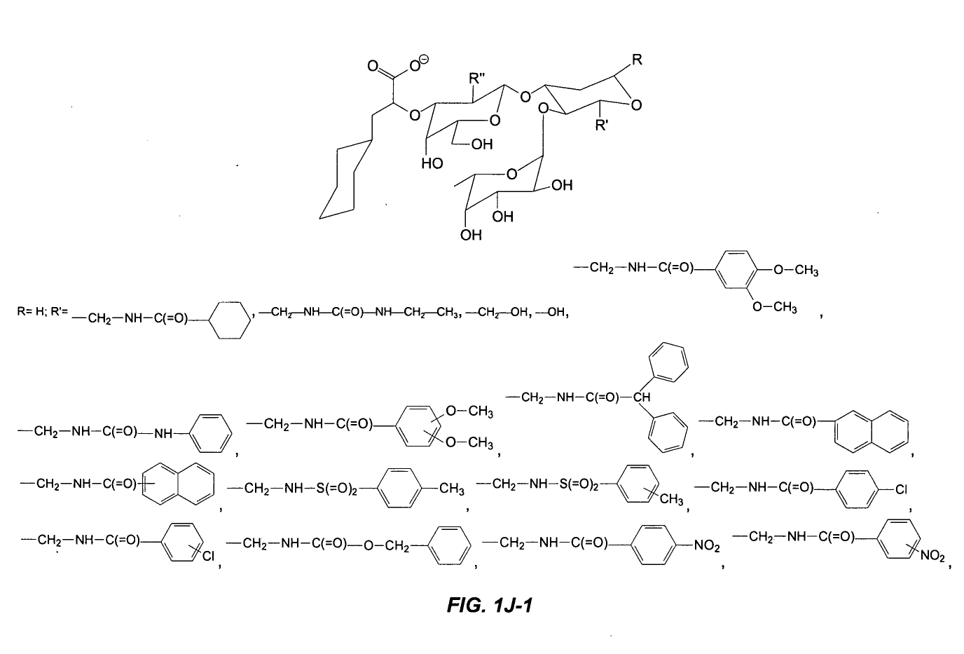

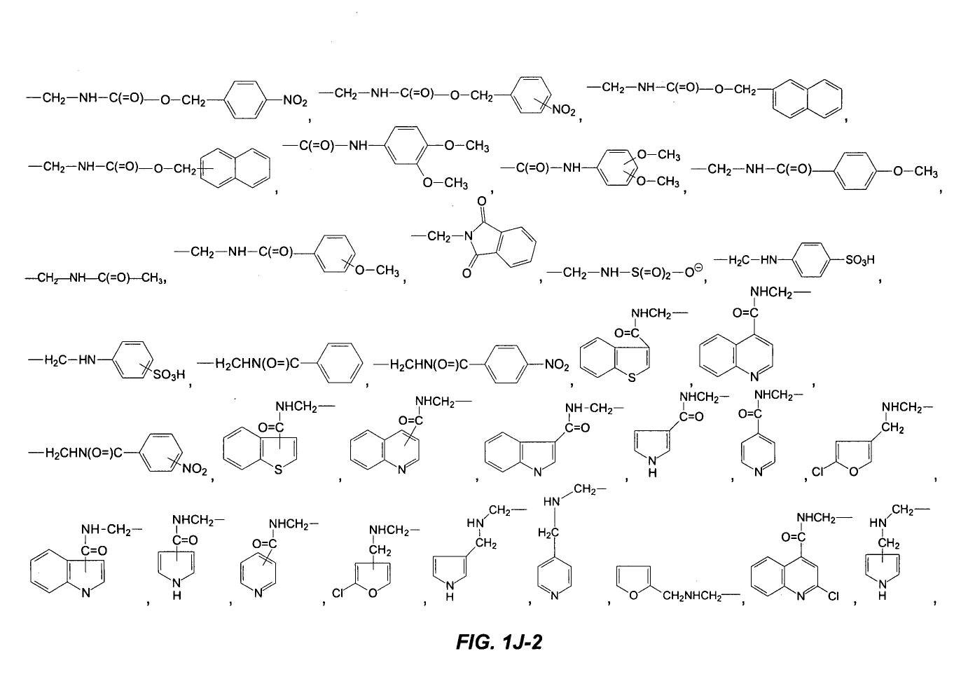

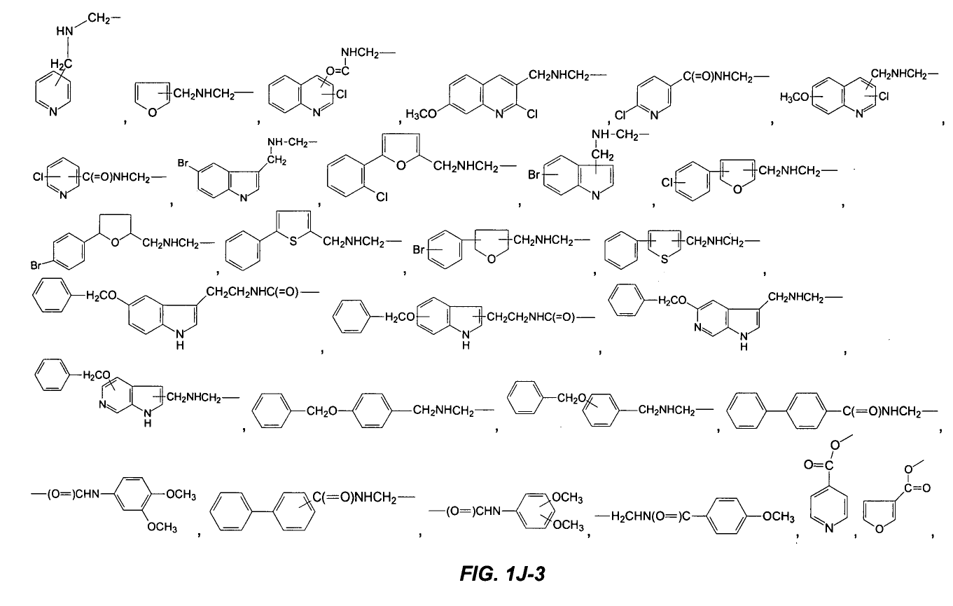

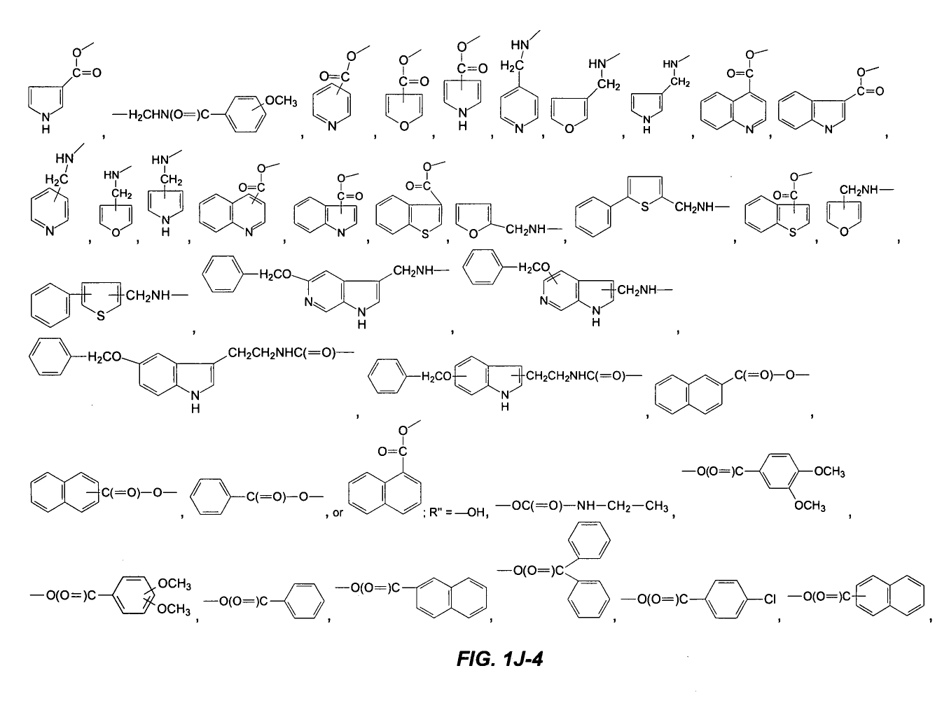

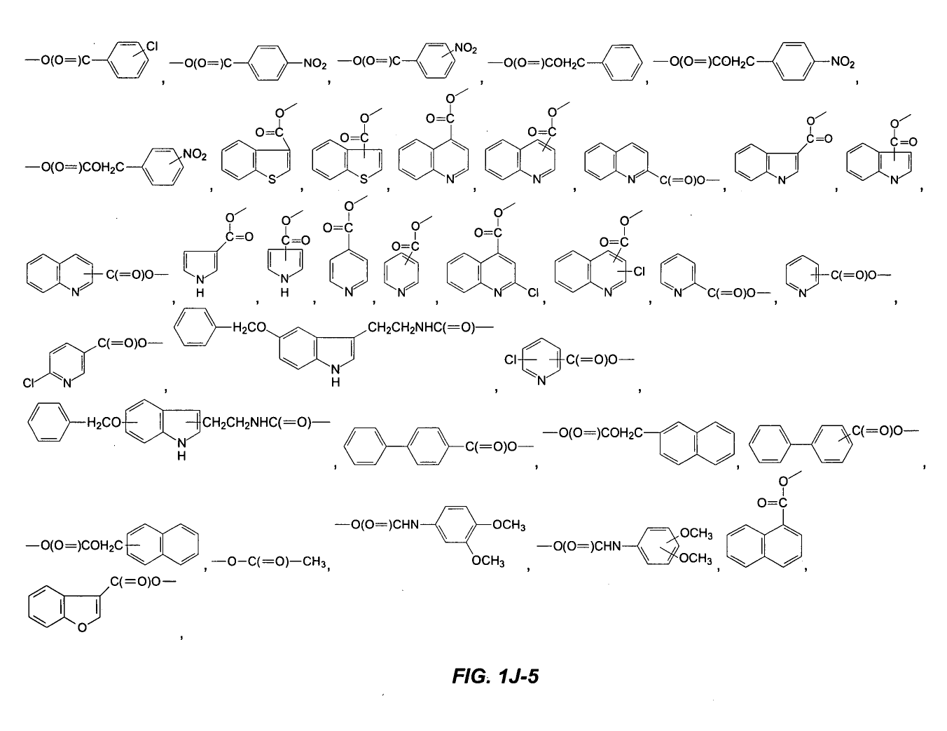

Representative BASA portions and analogues are included in the compounds shown in FIGS. 1A-1I. It will be apparent to those of ordinary skill in the art that modifications may be made to the compounds shown within these figures, without adversely affecting the ability to function as selectin modulators. Such modifications include deletions, additions and substitutions as described above.

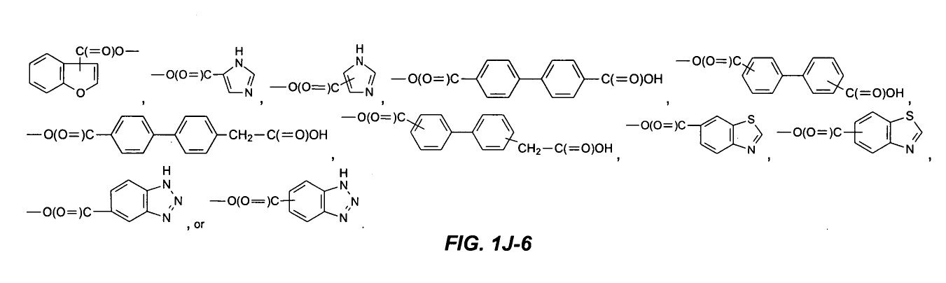

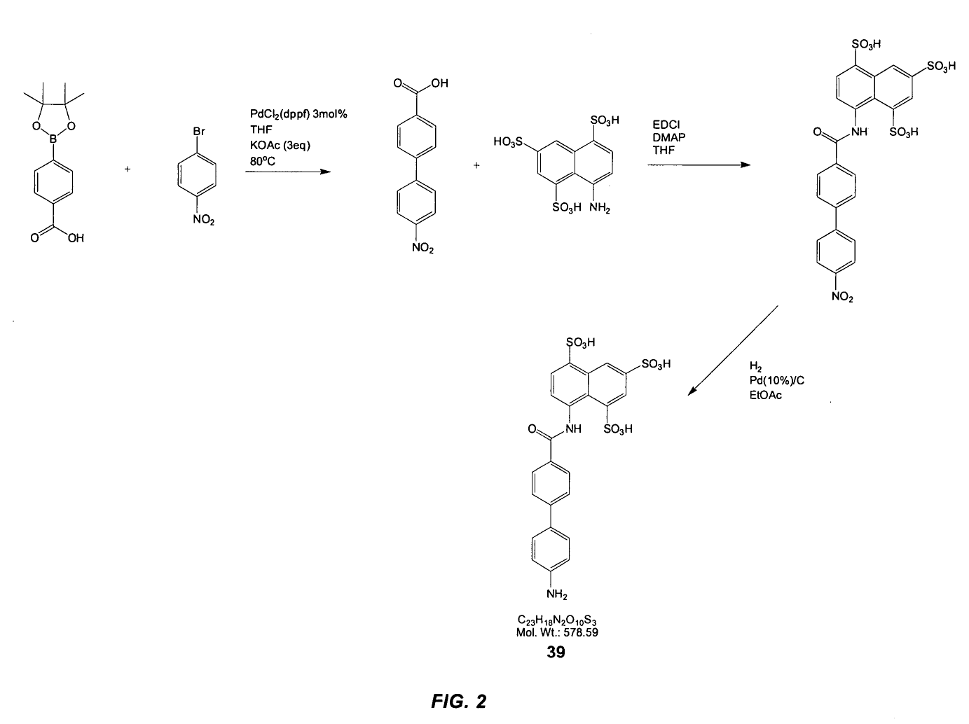

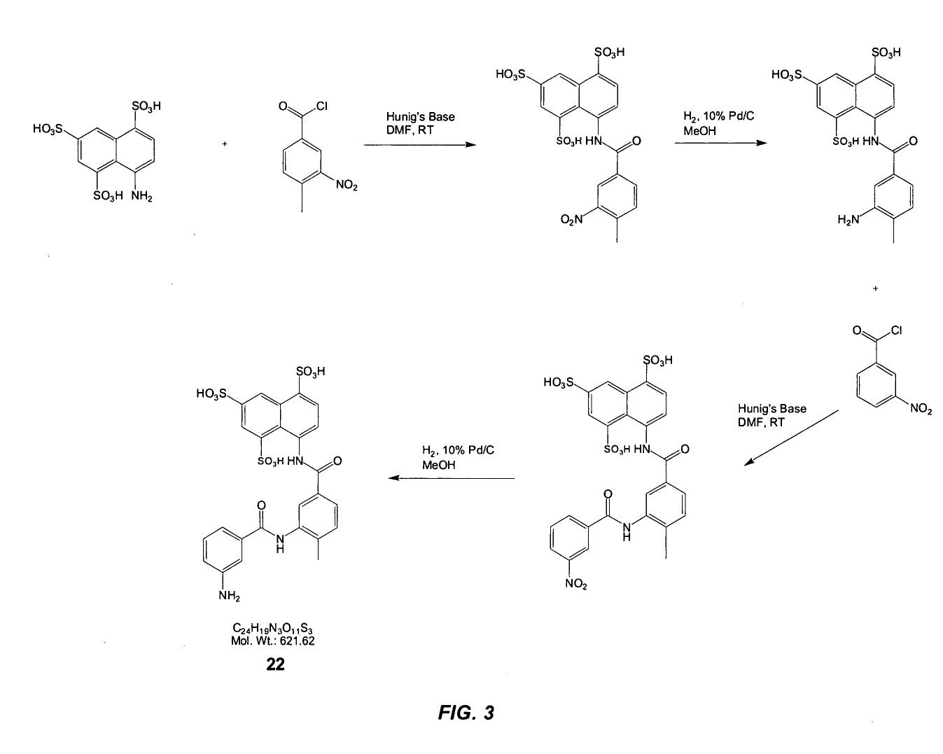

Certain selectin modulator components are commercially available from, for example, Sigma-Aldrich, Toronto Research Chemicals, Calbiochem and others. Others may be prepared using well known chemical synthetic techniques from available compounds. General synthetic methods for the synthesis of selectin modulators include the following: Amide formation of a primary or secondary amine or aniline can be accomplished via reaction with an acyl halide or carboxylic acid (see FIGS. 2 and 3). N-linked alkyl compounds are prepared by reductive amination of the amine/aniline with an aldehyde followed by imine reduction via sodium cyanoborohydride. Biphenyl compounds are easily prepared by reaction of suitable aryl bromide/iodides with appropriate boronic acids via Suzuki/Negishi conditions (see FIG. 2). Reduction of nitro groups can be selectively accomplished in the presence of other sensitive substrates by palladium catalyzed hydrogenation (see FIGS. 2 and 3).

A BASA component (such as those set forth above) is linked (e.g., covalently attached with or without a spacer group) to a particular selectin-binding glycomimetic (or glycoconjugate thereof) to form a selectin modulator of the present invention. Examples of preferred glycomimetics are shown in FIG. 1J. When a BASA is attached at R, R′ or R″ of FIG. 1J, the substituent listed for the particular position is typically replaced by the BASA.

The particular glycomimetics are generally: 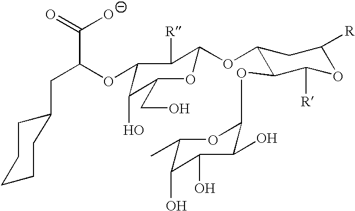
R, R′ and R″ are positions at which a BASA can be attached. Only a single BASA is attached to a particular glycomimetic (i.e., a BASA is attached at only one of R, R′ and R″ in a given molecule). When a BASA is not attached at R, the R substituent is hydrogen (H). When a BASA is not attached at R′, the R′ substituent is one of the substituents disclosed herein, or other aromatic substituents including other heteroaromatics, or other non-aromatic cyclic substituents including non-aromatic heterocycles. When a BASA is not attached at R″, the R″ substituent is one of the substituents disclosed herein or other aromatic substituents. Substituents other than —OH at R′ and R″ are preferred.

The attachment of a BASA to a particular glycomimetic can be accomplished in a variety of ways to form a selectin modulator. A linker possessed by (or added to) a BASA or a glycomimetic may include a spacer group, such as —(CH₂)ₙ— or —O(CH₂)ₙ— where n is generally about 1-20 (including any whole integer range therein). An example of a linker is —NH₂ on a glycomimetic, e.g., —CH₂—NH₂ when it includes a short spacer group. In an embodiment, —CH₂—NH₂ is attached to a glycomimetic at R′ which may then be used to attach a BASA. The simplest attachment method is reductive amination of the BASA to a glycomimetic containing a reducing end (an anomeric hydroxyl/aldehyde). This is accomplished by simple reaction of the BASA to the reducing end and subsequent reduction (e.g., with NaCNBH₃ at pH 4.0) of the imine formed. The most general approach entails the simple attachment of an activated linker to the glycomimetic via an O, S or N heteroatom (or C atom) at the anomeric position. The methodology of such attachments has been extensively researched for carbohydrates and anomeric selectivity is easily accomplished by proper selection of methodology and/or protecting groups. Examples of potential glycosidic synthetic methods include Lewis acid catalyzed bond formation with halogen or peracetylated sugars (Koenigs Knorr), trichloroacetamidate bond formation, thioglycoside activation and coupling, glucal activation and coupling, n-pentenyl coupling, phosphonate ester homologation (Horner-Wadsworth-Emmons reaction), and many others. Alternatively, linkers could be attached to positions on the moieties other than the anomeric. The most accessible site for attachment is at a six hydroxyl (6-OH) position of a glycomimetic (a primary alcohol). The attachment of a linker at the 6-OH can be easily achieved by a variety of means. Examples include reaction of the oxy-anion (alcohol anion formed by deprotonation with base) with an appropriate electrophile such as an alkyl/acyl bromide, chloride or sulfonate ester, activation of the alcohol via reaction with a sulfonate ester chloride or POCl₃ and displacement with a subsequent nucleophile, oxidation of the alcohol to the aldehyde or carboxylic acid for coupling, or even use of the Mitsunobu reaction to introduce differing functionalities. Once attached the linker is then functionalized for reaction with a suitable nucleophile on the BASA (or vice versa). This is often accomplished by use of thiophosgene and amines to make thiourea-linked heterobifunctional ligands, diethyl squarate attachment (again with amines) and/or simple alkyl/acylation reactions. Additional methods that could be utilized include FMOC solid or solution phase synthetic techniques traditionally used for carbohydrate and peptide coupling and chemo-enzymatic synthesis techniques possibly utilizing glycosyl/fucosyl transferases and/or oligosaccharyltransferase (OST).

Embodiments of linkers include the following: 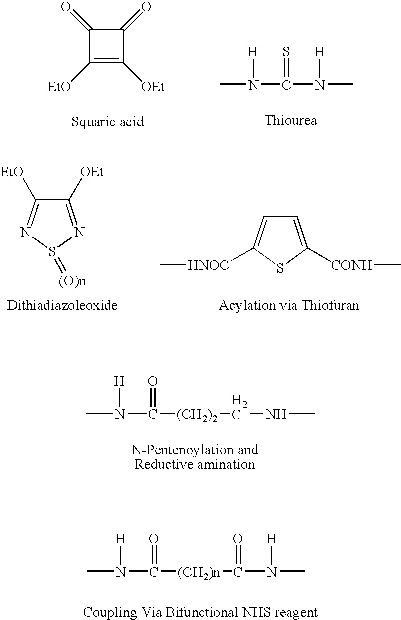
Other linkers will be familiar to those in the art.

Although selectin modulators as described herein may sufficiently target a desired site in vivo, it may be beneficial for certain applications to include an additional targeting moiety to facilitate targeting to one or more specific tissues. As used herein, a “targeting moiety,” may be any substance (such as a compound or cell) that, when linked to a modulating agent enhances the transport of the modulator to a target tissue, thereby increasing the local concentration of the modulator. Targeting moieties include antibodies or fragments thereof, receptors, ligands and other molecules that bind to cells of, or in the vicinity of, the target tissue. Linkage is generally covalent and may be achieved by, for example, direct condensation or other reactions, or by way of bi- or multi-functional linkers.

For certain embodiments, it may be beneficial to also, or alternatively, link a drug to a selectin modulator. As used herein, the term “drug” refers to any bioactive agent intended for administration to a mammal to prevent or treat a disease or other undesirable condition. Drugs include hormones, growth factors, proteins, peptides and other compounds. Examples of potential drugs include antineoplastic agents (such as 5-fluorouracil and distamycin), integrin agonist/antagonists (such as cyclic-RGD peptide), cytokine agonist/antagonists, histamine agonist/antagonists (such as diphenhydramine and chlorpheniramine), antibiotics (such as aminoglycosides and cephalosporins) and redox active biological agents (such as glutathione and thioredoxin). In other embodiments, diagnostic or therapeutic radionuclides may be linked to a selectin modulator. In many embodiments, the agent may be linked directly or indirectly to a selectin modulator.

Evaluating Inhibition of Selectin-Mediated Intercellular Adhesion

Modulating agents as described above are capable, for example, of inhibiting selectin-mediated cell adhesion. This ability may generally be evaluated using any of a variety of in vitro assays designed to measure the effect on adhesion between selectin-expressing cells (e.g., adhesion between leukocytes and platelets or endothelial cells). For example, such cells may be plated under standard conditions that, in the absence of modulator, permit cell adhesion. In general, a modulator is an inhibitor of selectin-mediated cell adhesion if contact of the test cells with the modulator results in a discernible disruption of cell adhesion. For example, in the presence of modulators (e.g., micromolar levels), disruption of adhesion between leukocytes and platelets and/or endothelial cells may be determined visually within approximately several minutes, by observing the reduction of cells interacting with one another.

Selectin Modulator Formulations

Modulators as described herein may be present within a pharmaceutical composition. A pharmaceutical composition comprises one or more modulators in combination with one or more pharmaceutically or physiologically acceptable carriers, diluents or excipients. Such compositions may comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Within yet other embodiments, compositions of the present invention may be formulated as a lyophilizate. Compositions of the present invention may be formulated for any appropriate manner of administration, including for example, topical, oral, nasal, intravenous, intracranial, intraperitoneal, subcutaneous, or intramuscular administration.

A pharmaceutical composition may also, or alternatively, contain one or more active agents, such as drugs (e.g., those set forth above), which may be linked to a modulator or may be free within the composition.

The compositions described herein may be administered as part of a sustained release formulation (i.e., a formulation such as a capsule or sponge that effects a slow release of modulating agent following administration). Such formulations may generally be prepared using well known technology and administered by, for example, oral, rectal or subcutaneous implantation, or by implantation at the desired target site. Carriers for use within such formulations are biocompatible, and may also be biodegradable; preferably the formulation provides a relatively constant level of modulating agent release. The amount of modulating agent contained within a sustained release formulation depends upon the site of implantation, the rate and expected duration of release and the nature of the condition to be treated or prevented.

Selectin modulators are generally present within a pharmaceutical composition in a therapeutically effective amount. A therapeutically effective amount is an amount that results in a discernible patient benefit, such as increased healing of a condition associated with excess selectin-mediated function (e.g., intercellular adhesion), as described below.

Selectin Modulator Methods of Use

In general, the modulating agents and compositions described herein may be used for enhancing or inhibiting a selectin-mediated function. Such enhancement or inhibition may be achieved in vitro and/or in vivo in a warm-blooded animal, preferably in a mammal such as a human, provided that a selectin-expressing cell is ultimately contacted with a modulator, in an amount and for a time sufficient to enhance or inhibit selectin-mediated function.

Within certain aspects, the present invention provides methods for inhibiting the development of a condition associated with a selectin-mediated function, such as intercellular adhesion. In general, such methods may be used to prevent, delay or treat such a condition. In other words, therapeutic methods provided herein may be used to treat a disease, or may be used to prevent or delay the onset of such a disease in a patient who is free of disease or who is afflicted with a disease that is not associated with a selectin-mediated function. For example, the therapeutic methods have uses that may include the arrest of cell growth, the killing of cells, the prevention of cells or cell growth, the delay of the onset of cells or cell growth, or the prolongation of survival of an organism.

A variety of conditions are associated with a selectin-mediated function. Such conditions include, for example, tissue transplant rejection, platelet-mediated diseases (e.g., atherosclerosis and clotting), hyperactive coronary circulation, acute leukocyte-mediated lung injury (e.g., adult respiratory distress syndrome (ARDS)), Crohn's disease, inflammatory diseases (e.g., inflammatory bowel disease), autoimmune diseases (MS, myasthenia gravis), infection, cancer (and metastasis), thrombosis, wounds (and wound-associated sepsis), burns, spinal cord damage, digestive tract mucous membrane disorders (gastritis, ulcers), osteoporosis, rheumatoid arthritis, osteoarthritis, asthma, allergy, psoriasis, septic shock, traumatic shock, stroke, nephritis, atopic dermatitis, frostbite injury, adult dyspnoea syndrome, ulcerative colitis, systemic lupus erythematosus, diabetes and reperfusion injury following ischaemic episodes. Selectin modulators may also be administered to a patient prior to heart surgery to enhance recovery. Other uses include for pain management and for undesirable angiogenesis, e.g., associated with cancer.

Selectin modulators of the present invention may be administered in a manner appropriate to the disease to be treated (or prevented). Appropriate dosages and a suitable duration and frequency of administration may be determined by such factors as the condition of the patient, the type and severity of the patient's disease and the method of administration. In general, an appropriate dosage and treatment regimen provides the modulating agent(s) in an amount sufficient to provide therapeutic and/or prophylactic benefit. Within particularly preferred embodiments of the invention, a selectin modulator may be administered at a dosage ranging from 0.001 to 100 mg/kg body weight, on a regimen of single or multiple daily doses. Appropriate dosages may generally be determined using experimental models and/or clinical trials. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

Selectin modulators may also be used to target substances to cells that express a selectin. Such substances include therapeutic agents and diagnostic agents. Therapeutic agents may be a molecule, virus, viral component, cell, cell component or any other substance that can be demonstrated to modify the properties of a target cell so as to provide a benefit for treating or preventing a disorder or regulating the physiology of a patient. A therapeutic agent may also be a prodrug that generates an agent having a biological activity in vivo. Molecules that may be therapeutic agents may be, for example, polypeptides, amino acids, nucleic acids, polynucleotides, steroids, polysaccharides or inorganic compounds. Such molecules may function in any of a variety of ways, including as enzymes, enzyme inhibitors, hormones, receptors, antisense oligonucleotides, catalytic polynucleotides, anti-viral agents, anti-tumor agents, anti-bacterial agents, immunomodulating agents and cytotoxic agents (e.g., radionuclides such as iodine, bromine, lead, palladium or copper). Diagnostic agents include imaging agents such as metals and radioactive agents (e.g., gallium, technetium, indium, strontium, iodine, barium, bromine and phosphorus-containing compounds), contrast agents, dyes (e.g., fluorescent dyes and chromophores) and enzymes that catalyze a calorimetric or fluorometric reaction. In general, therapeutic and diagnostic agents may be attached to a selectin modulator using a variety of techniques such as those described above. For targeting purposes, a selectin modulator may be administered to a patient as described herein. Since selectins are chemotactic molecules for endothelial cells involved in the formation of new capillaries during angiogenesis, a selectin modulator may be used to target a therapeutic agent for killing a tumor's vasculature. A selectin modulator may also be used for gene targeting.

Selectin modulators may also be used in vitro, e.g., within a variety of well known cell culture and cell separation methods. For example, modulators may be linked to the interior surface of a tissue culture plate or other cell culture support, for use in immobilizing selectin-expressing cells for screens, assays and growth in culture. Such linkage may be performed by any suitable technique, such as the methods described above, as well as other standard techniques. Modulators may also be used, for example, to facilitate cell identification and sorting in vitro, permitting the selection of cells expressing a selectin (or different selectin levels). Preferably, the modulator(s) for use in such methods are linked to a detectable marker. Suitable markers are well known in the art and include radionuclides, luminescent groups, fluorescent groups, enzymes, dyes, constant immunoglobulin domains and biotin. Within one preferred embodiment, a modulator linked to a fluorescent marker, such as fluorescein, is contacted with the cells, which are then analyzed by fluorescence activated cell sorting (FACS).

All compounds of the present invention or useful thereto, include physiologically acceptable salts thereof.

The following Examples are offered by way of illustration and not by way of limitation.

### EXAMPLES

The syntheses of certain of the glycomimetics used in the present invention are illustrated in the following references: Helvetica Chemica Acta Vol. 83, pp. 2893-2907 (2000) and Angew. Chem. Int. Ed. Vol. 40, No. 19, pp. 3644-3647 (2001).

### Example 1

### Preparation of a Representative BASA (FIG. 2)

Synthesis of 39:

Suzuki Coupling

4-(4,4,5,5-Tetramethyl-[1,3,2]dioxaborolan-2-yl)-benzoic acid (0.004 mol, 1 eq) and KOAc (0.012 mol, 3 eq) are placed in THF (25 ml) creating a slurry. PdCl₂(dppf) (0.00012 mol, 3 mol %) and p-bromo-nitrobenzene (0.005 mol, 1.2 eq) are then added to the solution with stirring and the solution is heated gently to 80° C. After 6 hrs the reaction is complete by TLC (20:1 CH₂Cl₂/CH₃OH). The reaction mixture is evaporated to dryness, dissolved in CH₂Cl₂ (30 ml) and washed with distilled water and saturated NaHCO₃. The resultant biphenyl compound is taken directly to the next step.

Carbodiimide Coupling

4′-Nitro-biphenyl-4-carboxylic acid (0.004 mol, 1 eq), dimethyl amino pyridine (1 crystal, cat.) and EDCl (0.0041 mol, 1.05 eq) are dissolved in DMF (or THF, 20 ml) and allowed to react at room temperature for 10 min. 8-Amino-naphthalene-1,3,5-trisulfonic acid is added to the reaction mixture with stirring and the reaction is allowed to proceed at room temperature under nitrogen for 48 hrs. The reaction mixture is then evaporated to dryness and purified by reverse phase chromatography (C18 column, 80/20 CH₃CN/H₂O-1% TFA to 50/50 CH₃CN/H₂O).

Hydrogenation

8-[(4′-Nitro-biphenyl-4-carbonyl)-amino]-naphthalene-1,3,5-trisulfonic acid (1 eq) and 10% Pd (10 mol %) on carbon are placed in EtOAc (or CH₃OH). The solution is degassed and an atmosphere of H₂ is generated within the reaction vessel. The reaction is allowed to proceed until the uptake of H₂ ceases and TLC indicates the disappearance of starting material (˜12 hrs). The palladium precipitate is removed by filtration through a bed of celite and the filtrate is evaporated to dryness giving compound 39.

### Example 2

### Preparation of a Representative BASA (FIG. 3)

Synthesis of 22:

Acid Chloride Coupling

8-Amino-naphthalene-1,3,5-trisulfonic acid (0.004 mol, 1 eq) and diisopropyl ethyl amine (6 eq) are placed in DMF (20 ml) and cooled to 0° C. 3-nitro-4-methyl benzoyl chloride (0.005 mol, 1.2 eq) is dissolved in DMF and added dropwise to the cooled solution over 10 min. The reaction is allowed to proceed at 0° C. for 3 hrs. The reaction mixture is washed with 0.1M HCl (25 ml), frozen and evaporated to dryness. The resultant syrup is used without purification in the next step.

Hydrogenation

8-(4-Methyl-3-nitro-benzoylamino)-naphthalene-1,3,5-trisulfonic acid (1 eq) and 10% Pd on carbon (10 mol %) are placed in CH₃OH. The solution is degassed and an atmosphere of H₂ is generated within the reaction vessel. The reaction is allowed to proceed until the uptake of H₂ ceases and TLC indicates the disappearance of starting material (12 hrs). The palladium precipitate is removed by filtration through a bed of celite and the filtrate is evaporated to dryness giving the reduced compound 8-(3-Amino-4-methyl-benzoylamino)-naphthalene-1,3,5-trisulfonic acid.

Acid Chloride Coupling

8-(3-Amino-4-methyl-benzoylamino)-naphthalene-1,3,5-trisulfonic acid (0.004 mol, 1 eq) and diisopropyl ethyl amine (6 eq) are placed in DMF (15 ml) and cooled to 0° C. 3-Nitro-benzoyl chloride (0.005 mol, 1.2 eq) is dissolved in DMF (5 ml) and added dropwise to the cooled solution over 10 min. The reaction is allowed to proceed at 0° C. for 3 hrs. The reaction mixture is washed with 0.1M HCl (25 ml) and evaporated to dryness. The compound is purified by reverse phase chromatography (C18 column, 80/20 CH₃CN/H₂O-1% TFA to 50/50 CH₃CN/H₂O).

Hydrogenation

8-(3-(3-nitro-benzamido)-4-methyl-benzoylamino)-naphthalene-1,3,5-trisulfonic acid (1 eq) is dissolved in MeOD and is added 10% Pd on carbon (10 mole %). The reaction mixture is then shaken under an atmosphere of hydrogen for 16 h. The palladium is removed by filtration through a bed of celite and the filtrate is evaporated to dryness giving compound 22.

### Example 3

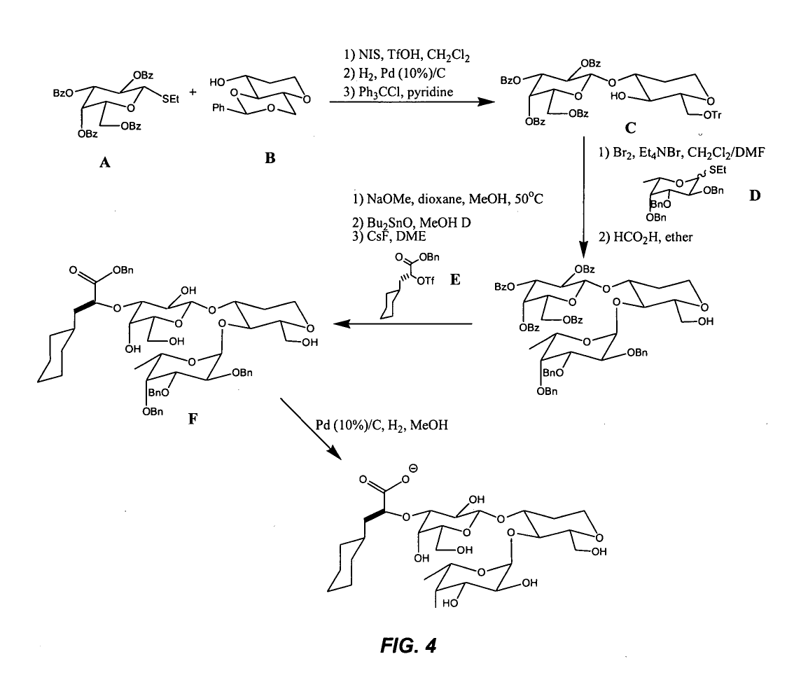

### Synthesis of Glycomimetic (FIG. 4)

Formation of Intermediate C:

Compound A (5.00 g, 12.74 mmol) and compound B (4.50 g, 19.11 mmol) and NIS (3.58 g, 15.93 mmol) are dissolved in CH₂Cl₂ (50 ml) and cooled to 0° C. A solution of trifluoromethanesulfonic acid (0.15 M in CH₂Cl₂) is added dropwise with stirring. After the solution changes color from orange to dark brown addition of TMS-OH ceases. The solution is then washed with saturated NaHCO₃ (30 ml) and the organic layer is dried with Na₂SO₄ and evaporated to dryness. The syrup obtained is purified by silica gel chromatography (hexane/ether, 1:1) and used in the next step.

The compound obtained previously is dissolved in THF (40 ml) and Pd (10%)/C (1/10 by mass) is added. The solution is degassed and an atmosphere of H₂ is generated. The reaction is allowed to proceed at RT until disappearance of starting material is confirmed by TLC. The solution is filtered thru a bed of celite and the filtrate is concentrated in vacuo giving the 4 and 60H compound. The compound is then dissolved in pyridine (25 ml) and cooled to 0° C. Ph₃CCl (1.2 eq) is added dropwise and the reaction is allowed to proceed at RT for 6 hrs. Ethyl acetate (50 ml) is then added and the solution is washed with 0.1N HCl (2×50 ml), saturated NaHCO₃ (1×50 ml) and saturated NaCl (1×50 ml). The organic layer is dried with Na₂SO₄ and evaporated to dryness. Intermediate C is obtained by silica gel chromatography.

Formation of Compound:

Compound C (800 mg, 1.41 mmol) and Et₄NBr (353 mg, 1.69 mmol) are dissolved in DMF/CH₂Cl₂ (10 ml, 1:1, containing molecular sieves) and cooled to 0° C. Br₂ (298 mg, 1.86 mmol, in CH₂Cl₂) is added dropwise to a separate solution of compound D (808 mg, 1.69 mmol) in CH₂Cl₂ at 0° C. After 30 min the Br₂/D solution is quenched with cyclohexene (0.2 ml) and added to the C solution immediately (within 10 min). This mixture is allowed to react for 65 hrs at RT. Ethyl acetate (100 ml) is added, the solution filtered, and the filtrate is washed with saturated NaS₂O₃ (2×50 ml) and saturated NaCl (2×50 ml). The organic layer is dried with Na₂SO₄ and evaporated to dryness. The resultant syrup is then dissolved in ether (50 ml) and formic acid (10 ml), is added with stirring. Upon completion of the reaction (as verified by TLC), the solution is washed with saturated NaHCO₃ (2×50 ml) and saturated NaCl (1×50 ml). The organic layer is dried with Na₂SO₄ then evaporated to dryness. The compound is then purified by silica gel chromatography.

Formation of Intermediate F:

The compound (1 g, 1.02 mmol) is dissolved in MeOH/dioxane (10 ml, 20:1) and NaOMe (0.10 mmol) is added with stirring. The reaction is allowed to proceed at 50° C. for 20 hrs and then 2 drops of acetic acid are added. The solution is evaporated to dryness, dissolved in ethyl ether (25 ml) and washed with saturated NaCl (1×50 ml). The organic layer is dried with Na₂SO₄ and evaporated to dryness. The final product is purified by silica gel chromatography. The product (0.980 mmol) and Bu₂Sn (1.08 mmol) are suspended in MeOH (15 ml) and heated to reflux for 2 hrs. The resultant clear solution is then evaporated to dryness, taken up in pentane (10 ml) and evaporated giving a colorless foam. The foam is dissolved in 1,2-dimethoxyethane (DME, 15 ml), compound E (1.96 mmol) and CsF (1.18 mmol) are added and the reaction stirred for 2 hrs at room temperature. After 2 hrs 1M KH₂PO₄ (50 ml) and KF (1 g) are added with stirring followed by extraction with ethyl acetate (2×25 ml). The organic layer is washed with 10% KF (2×50 ml) and saturated NaCl (2×50 ml), dried with Na₂SO₄ and evaporated to dryness under reduced pressure. Compound F is obtained via silica gel chromatography.

Formation of Glycomimetic:

Compound F is dissolved in CH₃OH (50 ml) and Pd (10%)/C (1/10 by mass) is added. The solution is degassed and an atmosphere of H₂ is generated. The reaction is allowed to proceed at RT until disappearance of starting material is confirmed by TLC. The solution is filtered thru a bed of celite and the filtrate is concentrated in vacuo giving the glycomimetic.

### Example 4

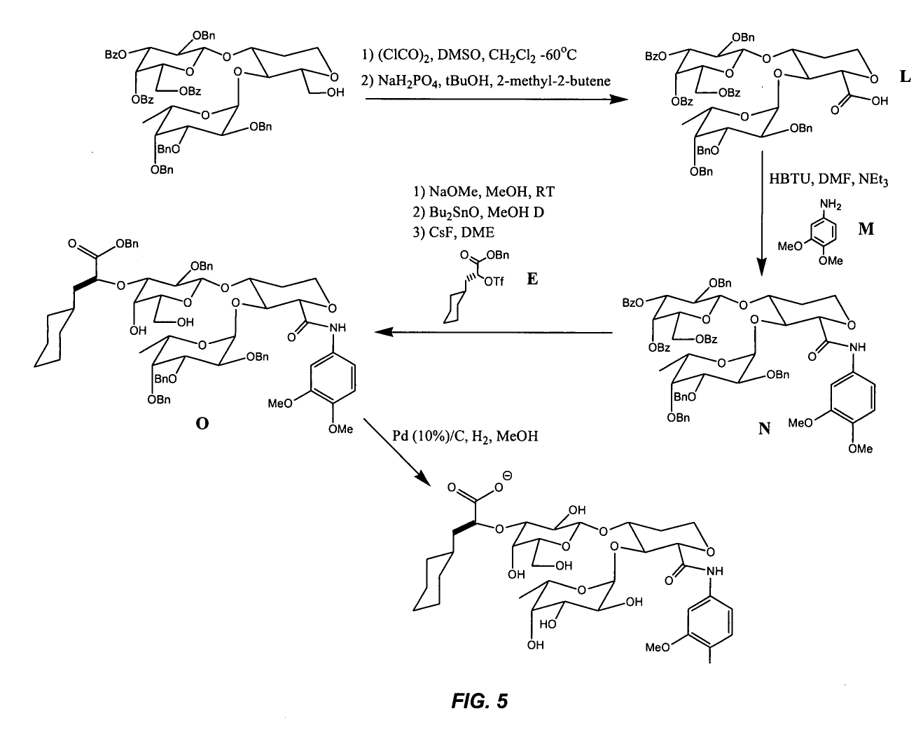

### Synthesis of Glycomimetic (FIG. 5)

Formation of Intermediate L:

The starting compound (10 mmol) is dissolved in CH₂Cl₂ (30 ml) and DMSO (20 mmol) is added and the solution is cooled to −60° C. Oxalyl chloride (11 mmol) is added slowly to the stirred solution of 20. The reaction is allowed to proceed for 30 min under N₂ atmosphere. The reaction is washed with 0.1M HCl, saturated NaHCO₃, and saturated NaCl. The organic layer is dried with Na₂SO₄ and evaporated to dryness. The resultant syrup is placed in tBuOH (20 ml) and 2-methyl-2-butene (10 ml) and NaH₂PO₄ (20 mmol) is added with stirring. The reaction is allowed to proceed for 3 hrs and is then evaporated taken up in CH₂Cl₂ and washed with 0.1M HCl, saturated NaHCO₃, and saturated NaCl. The resultant compound is purified by silica gel chromatography giving compound L.

Formation of Intermediate N:

Compound L (10 mmol) is dissolved in DMF (15 ml) and compound M (10 mmol), HBTU (12 mmol) and Et₃N (20 mmol) are added with stirring. The reaction is allowed to proceed at RT for 24 hrs. Ethyl acetate (100 ml) is added and the solution is washed with 0.1M HCl (1×100 ml), saturated NaHCO₃ (1×100 ml), and saturated NaCl (1×100 ml). The organic layer is dried with Na₂SO₄ and evaporated to dryness. Compound N is isolated via silica gel chromatography.

Formation of Intermediate O:

Compound N (10 mmol) is dissolved in MeOH (35 ml) and NaOMe (1 mmol) is added with stirring. The reaction is allowed to proceed at RT for 20 hrs. The solution is evaporated to dryness, dissolved in ethyl ether (50 ml) and washed with saturated NaCl (1×50 ml). The organic layer is dried with Na₂SO₄ and evaporated to dryness. The final product is purified by silica gel chromatography. The product (0.980 mmol) and Bu₂Sn (1.08 mmol) are suspended in MeOH (15 ml) and heated to reflux for 2 hrs. The resultant clear solution is then evaporated to dryness, taken up in pentane (10 ml) and evaporated giving a colorless foam. The foam is dissolved in 1,2-dimethoxyethane (DME, 15 ml), compound E (1.96 mmol) and CsF (1.18 mmol) are added and the reaction stirred for 2 hrs at room temperature. After 2 hrs 1M KH₂PO₄ (50 ml) and KF (1 g) are added with stirring followed by extraction with ethyl acetate (2×25 ml). The organic layer is washed with 10% KF (2×50 ml) and saturated NaCl (2×50 ml), dried with Na₂SO₄ and evaporated to dryness under reduced pressure. Compound O is obtained via silica gel chromatography.

Formation of Glycomimetic:

Compound O (9 mmol) is dissolved in MeOH (200 ml) and Pd (10%)/C (3 g) is added. The solution is degassed and an atmosphere of H₂ is generated. The reaction is allowed to proceed at RT until disappearance of starting material is confirmed by TLC. The solution is filtered thru a bed of celite and the filtrate is concentrated in vacuo giving the glycomimetic.

### Example 5

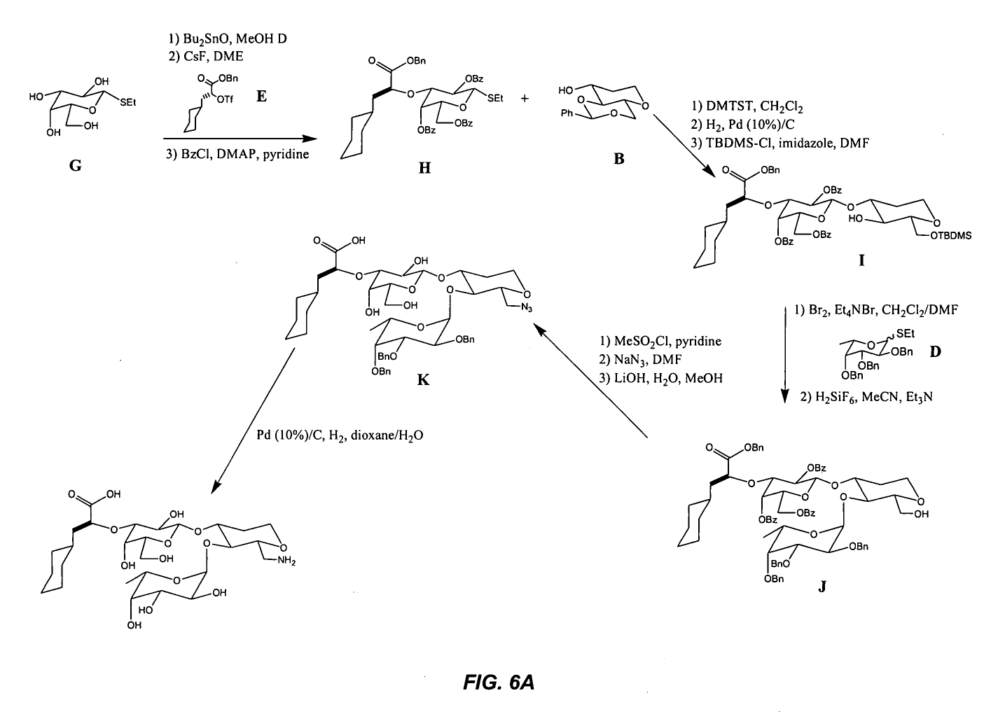

### Synthesis of Glycomimetic Precursor (FIG. 6A)

Formation of Intermediate H:

Compound G (15.0 g, 66.9 mmol) and Bu₂SnO (20.0 g, 80.3 mmol) are suspended in MeOH (450 ml) and heated to reflux for 2 hrs. The resultant clear solution is then evaporated to dryness, taken up in pentane and evaporated again giving a colorless foam. The foam is dissolved in 1,2-dimethoxyethane (DME, 120 ml), E (39.6 g, 100.3 mmol) and CsF (12.2 g, 80.3 mmol) are added and the reaction stirred for 2 hrs at room temperature. After 2 hrs 1M KH₂PO₄ (700 ml) and KF (25 g) are added with stirring followed by extraction with ethyl acetate (3×250 ml). The organic layer is washed with 10% KF (2×250 ml) and sat. NaCl (1×250 ml), dried with Na₂SO₄ and evaporated to dryness under reduced pressure. The compound (19.3 g, 41.2 mmol) is purified by silica gel chromatography and immediately dissolved in pyridine (210 ml) with a crystal DMAP. The solution is cooled to 0° C. and benzoyl chloride (52.1 g, 370.7 mmol) is added dropwise with stirring. The solution is allowed to warm to room temperature slowly and the reaction proceeds at RT for 20 min. The solution is evaporated to dryness, dissolved in ethyl acetate (500 ml), and washed with 0.1M HCl (2×250 ml), saturated NaHCO₃ (2×250 ml) and saturated NaCl (1×250 ml) solutions. The organic layer is dried with Na₂SO₄ and evaporated to dryness. H is obtained via silica gel chromatography.

Formation of Intermediate I:

Intermediate H (10.0 g, 12.82 mmol) and intermediate B (6.05 g, 25.64 mmol) are dissolved in CH₂Cl₂ (75 ml) and 0.15M CF₃SO₃H (in CH₂Cl₂) is added dropwise at −10° C. with stirring. Addition is stopped when the orange solution changes to brown. Ethyl acetate (500 ml) is added and the solution is washed with saturated NaHCO₃ (4×250 ml) and saturated NaCl (250 ml). The organic layer is then dried with Na₂SO₄ and evaporated under reduced pressure. The compound (7.96 g, 9.19 mmol) is then purified by silica gel chromatography and then dissolved in DMF (55 ml). TBDMS-Cl (1.52 g, 10.1 mmol) and imidazole (0.94 g, 13.8 mmol) are then added and the reaction allowed to proceed at RT for 1 hr. Ethyl acetate (250 ml) is added and the solution washed with saturated NaHCO₃ (5×250 ml) and saturated NaCl (1×250 ml). The organic layer is then dried with Na₂SO₄ and purified by silica gel chromatography giving intermediate 1.

Formation of Intermediate J:

Compound I (7.71 g, 7.87 mmol) and Et₄NBr (2.00 g, 9.45 mmol) are dissolved in DMF/CH₂Cl₂ (60 ml, 1:1, containing molecular sieves-12 g) and cooled to 0° C. Br₂ (1.90 g, 11.8 mmol) in CH₂Cl₂ (11 ml) is added dropwise to a separate solution of compound D (4.5 g, 9.45 mmol) in CH₂Cl₂ at 0° C. After 30 min the Br₂/D solution is quenched with cyclohexene (2.5 ml) and added to the I solution immediately (within 10 min). This mixture is allowed to react for 65 hrs at RT. CH₂Cl₂ (250 ml) is added, the solution filtered, and the filtrate is washed with saturated NaHCO₃ (2×50 ml), 0.5M HCl (2×250 ml) and saturated NaCl (2×250 ml). The organic layer is dried with Na₂SO₄ and evaporated to dryness. The mixture is dissolved in MeCN (85 ml) at RT and a solution of Et₃N (0.21 ml) and H₂SiF₆ (1.3 ml, 35%) in MeCN (17 ml) is added and stirred for 2 hrs. CH₂Cl₂ (250 ml) is added and the solution washed with saturated NaHCO₃ (3×250 ml) and saturated NaCl (1×250 ml). The organic layer is dried with Na₂SO₄, evaporated to dryness and J is purified by silica gel chromatography.

Formation of Intermediate K:

Intermediate J (12.5 g, 9.75 mmol) is dissolved in pyridine (80 ml) and methanesulfonylchloride (3.35 g, 29.2 mmol) is added dropwise with stirring over 5 min. The reaction is allowed to proceed for 30 min and then ethyl acetate (500 ml) is added. The solution is washed with 1N HCl (250 ml). The organic layer is dried with Na₂SO₄ and evaporated. The resultant syrup (12.95 g, 9.52 mmol) is dissolved in DMF (40 ml) and NaN₃ (4.64 g, 74.4 mmol) is added. The reaction is allowed to proceed for 35 hrs under argon atmosphere at 65° C. The solution is diluted with ethyl acetate (500 ml) and washed with H₂O (300 ml) and saturated NaCl (150 ml). The organic layer is dried with Na₂SO₃ and evaporated to dryness. The compound is purified by silica gel chromatography. The purified product (12.2 g, 9.33 mmol) is then suspended in MeOH/H₂O (200 ml/20 ml) solution and LiOH—H2O (5.1 g, 121.3 mmol) is added. The reaction is allowed to proceed at 65° C. for 20 hrs. Ethyl ether (500 ml) is added and the solution is washed with saturated NaCl (200 ml). The organic layer is dried with Na₂SO₄ and evaporated to dryness. Compound K is purified via silica gel chromatography.

Formation of Glycomimetic Precursor:

Compound K (8.45 g, 9.33 mmol) is dissolved in dioxane/H₂O (250 ml/50 ml) and Pd (10%)/C (3.4 g) is added. The solution is degassed and an atmosphere of H₂ is generated. The reaction is allowed to proceed at RT until disappearance of starting material is confirmed by TLC. The solution is filtered thru a bed of celite and the filtrate is concentrated in vacuo giving the glycomimetic precursor.

### Example 6

### Synthesis of Glycomimetics (FIG. 6B)

The glycomimetic precursor used in this Example is described in Example 5 (FIG. 6A).

Reaction of Glycomimetic Precursor With Acid Chlorides:

The glycomimetic precursor (20 mg, 0.033 mmol) is dissolved in a THF/H₂O (2 ml, 1:1) solution containing 1N NaOH (pH adjusted between 8-10) and is cooled to 0° C. Cyclohexyl-carbonylchloride (0.049 mmol) is then added dropwise with stirring. The reaction is allowed to continue at 0° C. for 3 hrs. The solution is quenched with ice and the solution is evaporated to dryness. The glycomimetic is purified by reverse phase chromatography.

Reaction of Glycomimetic Precursor With Isocyanates:

The glycomimetic precursor (30 mg, 0.049 mmol) is dissolved in a 0.5N aqueous NaOH solution (1 ml) and cooled to 0° C. Ethyl isocyanate (1.2 eq) is then added dropwise with stirring. The reaction is allowed to continue at RT for 3 hrs. The solution is quenched with ice and the solution is evaporated to dryness. The glycomimetic is purified by reverse phase chromatography.

Reaction of Glycomimetic Precursor With Chloro-Orthoformates:

The glycomimetic precursor (20 mg, 0.033 mmol) is dissolved in a THF/H₂O (2 ml, 1:1) solution containing NaOH (pH adjusted between 8-10) and is cooled to 0° C. Benzyl-chloro-orthoformate (0.049 mmol) is then added dropwise with stirring. The reaction is allowed to continue at 0° C. for 3 hrs. The solution is quenched with ice and the solution is evaporated to dryness. The glycomimetic is purified by reverse phase chromatography.

Reaction of Glycomimetic Precursor With Sulfonyl Chlorides:

The glycomimetic precursor (20 mg, 0.033 mmol) is dissolved in a saturated aqueous NaHCO₃/toluene (2 ml, 1:1) solution and is cooled to 0° C. p-Toluenesulfonyl chloride (0.049 mmol) is then added dropwise with stirring. The reaction is allowed to continue at 0° C. for 3 hrs. The solution is quenched with ice and the solution is evaporated to dryness. The glycomimetic is purified by reverse phase chromatography.

### Example 7

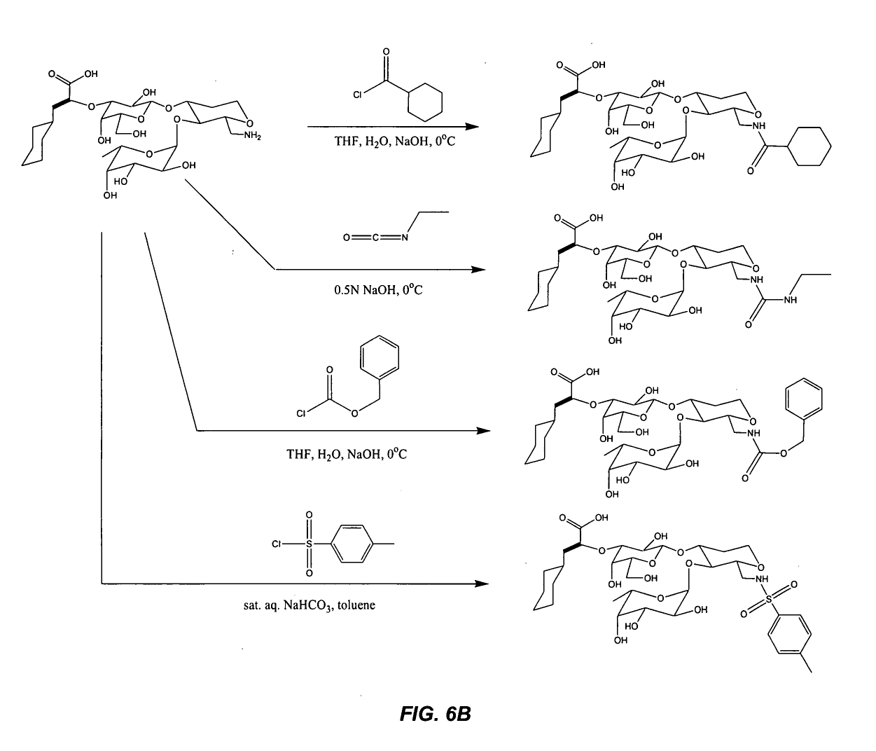

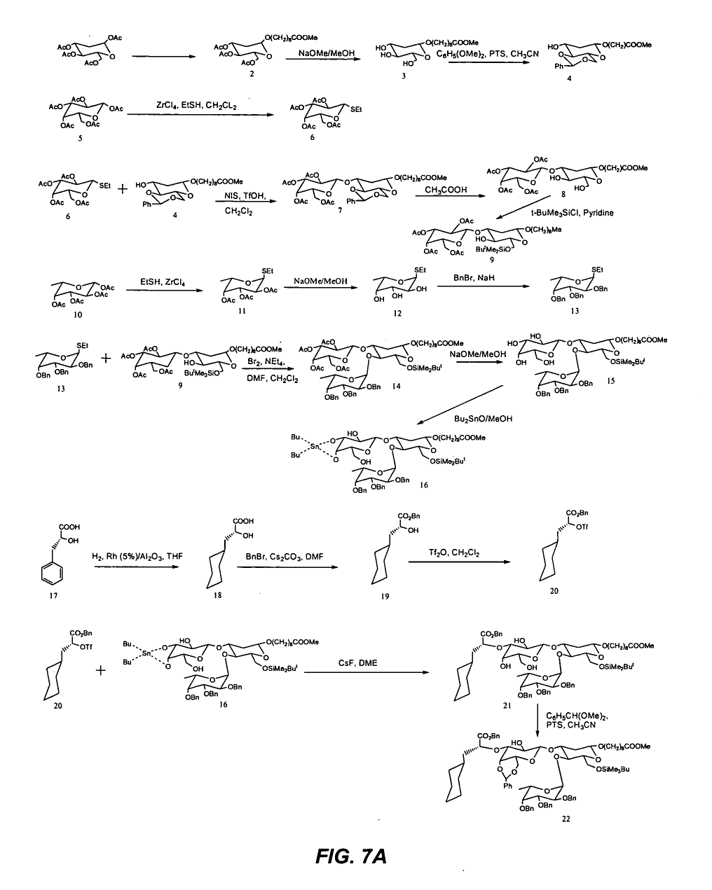

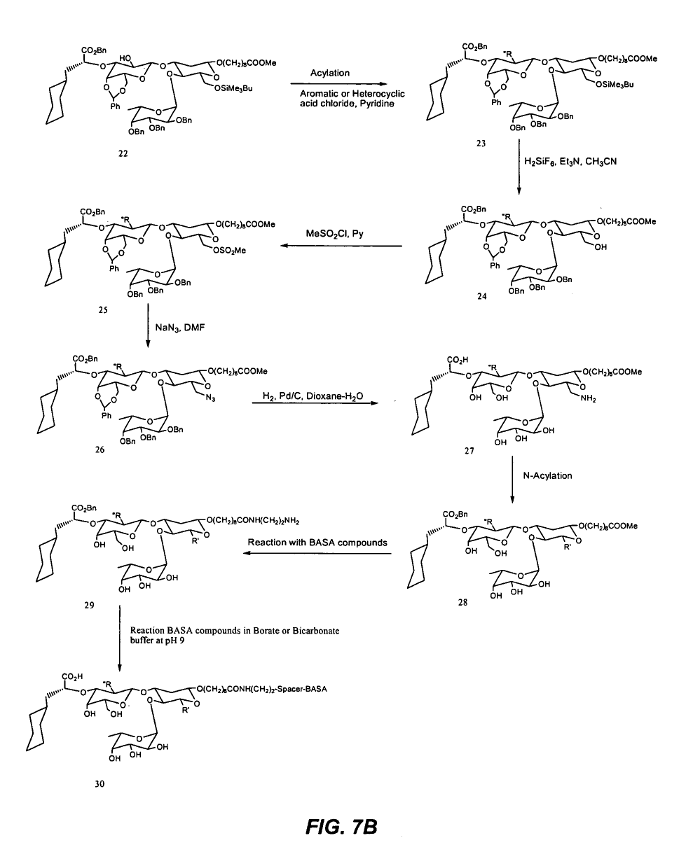

### Synthesis of Glycomimetic-BASA (FIGS. 7A and 7B)

Synthesis of Compound 4:

Starting from commercially available 2-deoxy glucose (15 g), compound 4 is synthesized following the procedure described in the literature (Bioorg. Med. Chem. Lett. 11, 2001, 923-925; Carbohydr. Res. 197, 1990, 75).

Synthesis of Compound 6:

Compound 6 is synthesized from commercially available 5 (25 g) as described in the literature (Carbohydr. Res., 193, 1989, 283-287).

Synthesis of Compound 9:

Compound 4 (5 g) is dissolved in dichloromethane (100 ml) and N-iodosucinimide (NIS, 10 g) and compound 6 (7.5 g) are added. The mixture is stirred at room temperature for 30 min with molecular sieves (4 A). The reaction mixture is cooled down to 0-5 degree and trifluoromethanesulfonic acid (0.05 M) in dichloromethane is added dropwise during 1 h and the reaction mixture is continued to stir at 0-5 degree for 2 h. Molecular sieves are filtered off through a celite bed and organic layer is extracted with water, saturated solution of sodium bicarbonate and water. Silica gel chromatography of the crude reaction mixture gives compound 7 in 75% yield.

Compound 7 (7 g) is treated with 80% acetic acid in water at 80 degrees centigrade for 2 h. Solvent is removed by evaporation to give 8 in 92% yield.

Compound 8 (6 g) is dissolved in DMF (60 ml) and 1H-imidazole, tert-butyl-trimethyl-silyl chloride (4 ml) is added. The reaction mixture is stirred at room temperature for 1 h. The reaction mixture is diluted with ethyl acetate and washed with water, and saturated solution of sodium bicarbonate. The organic layer is evaporated to dryness to give 9 in 90% yield.

Synthesis of Compound 13:

Compound 13 (12 g) is prepared following the procedure as described in the literature (Carbohydr. Res. 201, 1990, 15-30).

Synthesis of Compound 16:

To a solution of compound 13 (4 g) and compound 9 (4 g) in dichloromethane-DMF is added molecular sieves (4 A) and tetraethyl ammonium bromide and the mixture is stirred for 1 h at room temperature (RT). A solution of bromine (0.2 g) in dichloromethane (10 ml) is added dropwise with stirring at RT. Stirring is continued for 2 h at RT. The reaction mixture is filtered off through a bed of celite and the organic layer is washed with water and a saturated solution of sodium bicarbonate. Solvent is removed by evaporation and the syrupy residue is subjected to silica gel chromatography to give 14 in 70% yield.

Compound 14 is treated with 0.01M NaOMe/MeOH for 2 h to give 15 in 96% yield.

Compound 15 (4 g) is treated with dibutyltinoxide in MeOH under refluxing condition for 4 h. Solvent is removed by evaporation to give crude 16.

Synthesis of Compound 20:

Starting from commercially available phenyllactic acid compound 17 is synthesized as described (J. Med. Chem. 42, 1999, 4909-4913).

Synthesis of Compound 23:

Compound 16 (7 g crude) and compound 20 (3 g) are dissolved in Dimethoxyethane (DME) and CsF (1 g) is added. The resulting mixture is stirred at RT for 8 h. Water is added to the reaction mixture and is extracted with ethyl acetate. The organic layer is evaporated to dryness and the residue is purified by silicagel chromatography to give 21 in 64% overall yield.

To a suspension of compound 21 (3.5 g) in acetonitrile (100 ml) is added a,a-dimethoxytoluene (0.5 ml) and p-toluene-sulfonic acid (0.2 g). The reaction mixture is stirred at RT for 4 h. Triethylamine (0.4 ml) is added and solvent is removed by evaporation. The residual mixture is purified by silica gel chromatography to give compound 22 in 88% yield.

For the synthesis of O-acylated compounds in general, compound 22 (1 g each) is dissolved in pyridine (15 ml) and acyl chloride (aromatic and heterocyclic acid chloride) is added. The reaction mixture is stirred at RT for 2 h and then solvent removed by evaporation. The residue is purified by silica gel chromatography to give the corresponding acylated derivative 23 in 80-92% yield.

Synthesis of Compound 24:

Compound 23 (1 g) is dissolved in acetonitrile (25 ml) and to the solution is added triethylamine (0.1 ml). H₂SiF₆ (0.5 ml) in acetonitrile (5 ml) is added and the reaction mixture is stirred at RT for 2 h. The reaction mixture is diluted with dichloromethane and washed successively with water, a saturated solution of sodium bicarbonate, and water. The organic layer is evaporated to dryness and purified by silica gel chromatography to give compound 24 in 75% yield.

Synthesis of Compound 25:

To a solution of compound 24 (0.8 g) in dry pyridine (10 ml) is added dropwise a solution of methanesulfonylchloride (0.3 ml) with stirring at RT. After 30 min, the mixture is diluted with EtOAc and washed successively with water, saturated solution of sodium bicarbonate and water. The organic layer is removed by evaporation to dryness and the residue is purified by silica gel chromatography to give 25 in 95% yield.

Synthesis of Compound 26:

To a solution of compound 24 (0.7 g) in DMF (5 ml), sodium azide (0.3 g) is added. The mixture is heated at 65 degrees under argon and stirred for 28 h. After cooling to RT, EtOAc (44 ml) is added and washed with water. The organic layer is evaporated to dryness and purified by silica gel chromatography to give 26 in 96% yield.

Synthesis of Compound 27:

To a solution of compound 26 (0.5 g) in dioxane-water (5:1, 12 ml) is added 10% Pd—C (0.2 g) and the reaction mixture is stirred vigorously for 22 h under an atmosphere of hydrogen. The reaction mixture is filtered through a bed of celite and solvent is removed by evaporation. The residue is purified by silicagel chromatography to give 27 in 77% yield.

Synthesis of 28:

To a solution of compound 27 (50 mg) in THF/Water 1:1 (5 ml) is added commercially available acid chloride (0.1 g) in THF (0.5 ml). The pH of the reaction mixture is adjusted to 8-10 by the addition of 1N NaOH and maintained throughout the reaction. If necessary, additional acid chloride is added after 1-4 h, and after a total of 2-42 h, the mixture is partially evaporated to remove THF. Water is removed by evaporation, and the reaction mixture is purified by silica gel chromatography to yield N-acylated compounds in 77-88% yield.

Synthesis of Compound 30:

Compound 28 is first reacted with ethylene diamine and the resulting derivative 29 is obtained in 80% yield after silica gel chromatography. Compound 29 is reacted with BASA compounds with suitable spacer (such as, for example, squaric acid, isothiocyanates, isocyanates, histidine, disuccinimidyl glutarate) at pH 9 to give corresponding glycomimetics linked to BASA (Compound 30).

### Example 8

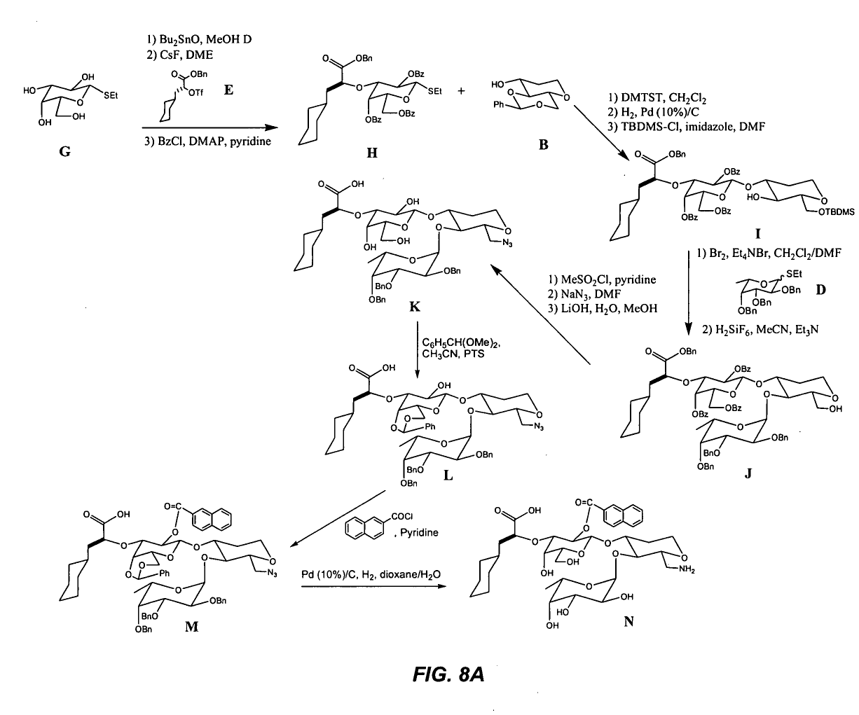

### Synthesis of Glycomimetics (FIGS. 8A and 8B)

Formation of Intermediate L:

Compound K (1 g) (prepared according to Example 5) is dissolved in acetonitrile and treated with a,a-dimethoxy toluene in the presence of p-toluene-sulfonic acid for 4 h at room temperature. The reaction mixture is neutralized with triethylamine and concentrated to dryness. The reaction mixture is then purified by silica-gel chromatography to give pure compound L.

Formation of Intermediate M:

Compound L (1 g) is treated with naphthoyl chloride in pyridine for 16 h. The crude reaction mixture is diluted with dichloromethane and the organic layer is washed successively with cold 0.1N HCl, cold saturated solution of sodium bicarbonate and cold brine solution. The organic layer is dried over sodium sulfate and concentrated to dryness. The resulting product is purified by silicagel chromatography to give compound M in 80% yield.

Formation of Compound N:

To a solution of compound M (1 g) in dioxane-water is added 10% palladium on carbon and the suspension is shaken at room temperature for 48 h under a positive pressure of hydrogen. Catalyst is filtered off through a bed of celite and the solution is concentrated to dryness to give compound N.

Synthesis of Glycomimetics (FIG. 8B):

The glycomimetic precursor N is reacted with acid chlorides, isocyanates, chloro-orthoformates, or sulfonyl chlorides using the procedures described in Example 6.

### Example 9

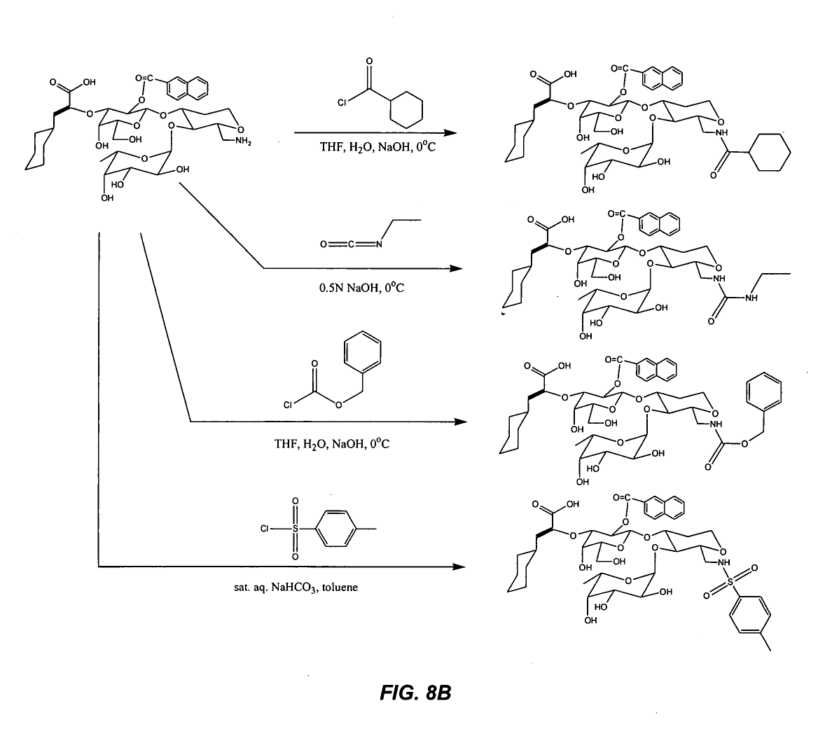

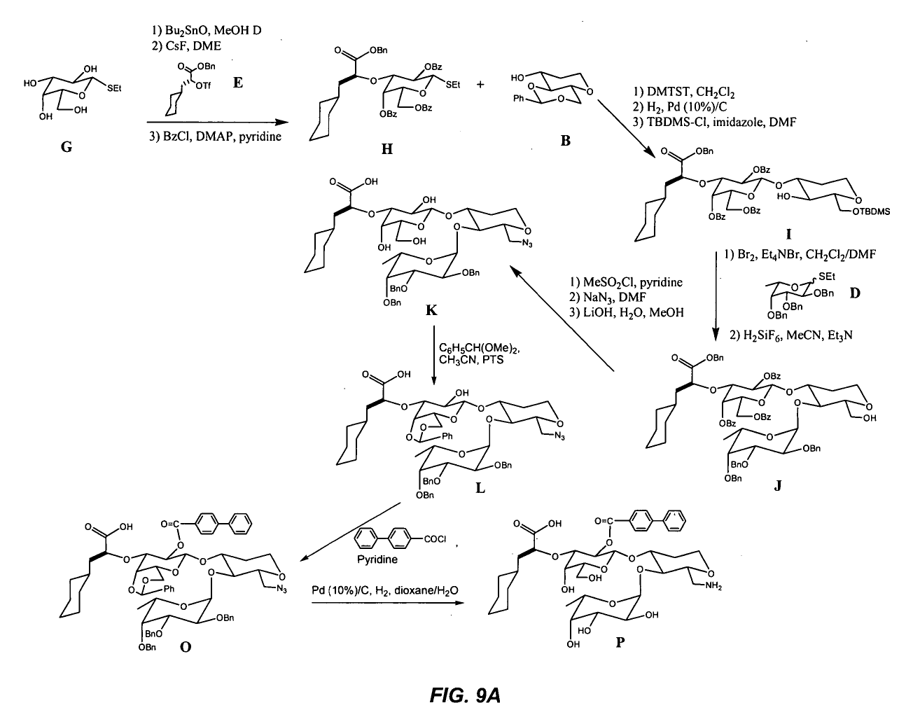

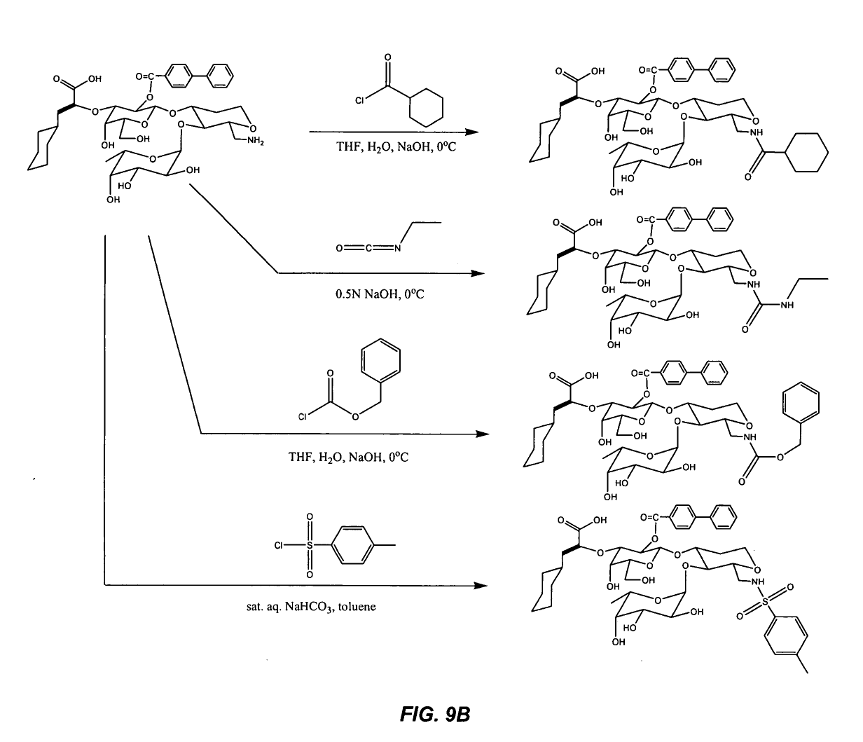

### Synthesis of Glycomimetics (FIGS. 9A and 9B)

Formation of Intermediate O:

Compound L (1 g) (prepared according to Example 8) is treated with 4-phenyl-benzoyl chloride exactly the same way as described for intermediate M (Example 8) and purified by silicagel chromatography.

Formation of Compound P:

Compound O is hydrogenated with 10% palladium on carbon exactly the same as described for compound N (Example 8) to afford compound P.

Synthesis of Glycomimetics (FIG. 9B):

The glycomimetic precursor P is reacted with acid chlorides, isocyanates, chloro-orthoformates, or sulfonyl chlorides using the procedures described in Example 6.

### Example 10

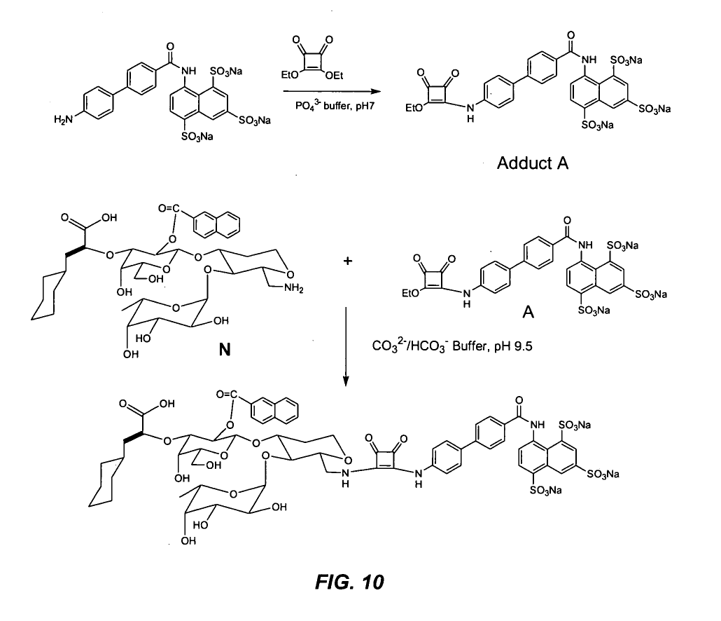

### Synthesis of Glycomimetic-BASA (FIG. 10)

Condensation Between BASA and Diethyl Squarate:

The BASA of Example 1 (10 mg) is reacted with diethyl squarate (5 mg) in phosphate buffer at pH 7 and then purified by preparative hplc to give the adduct A.

Synthesis of Glycomimetic N:

Glycomimetic N is synthesized as described in Example 8.

Condensation Between Glycomimetic N and Intermediate A:

To a solution of intermediate A (15 mg) in carbonate/bicarbonate buffer (pH 9.5, 1.5 ml) is added Glycomimetic N (10 mg) and the reaction mixture is stirred at room temperature for 16 h. The reaction mixture is then applied to column of sephadex G-25 and the column is eluted with 5 mM ammonium bicarbonate solution. The fractions that correspond to the product are collected and lyophilized to yield Glycomimetic-BASA conjugate (12 mg).

### Example 11

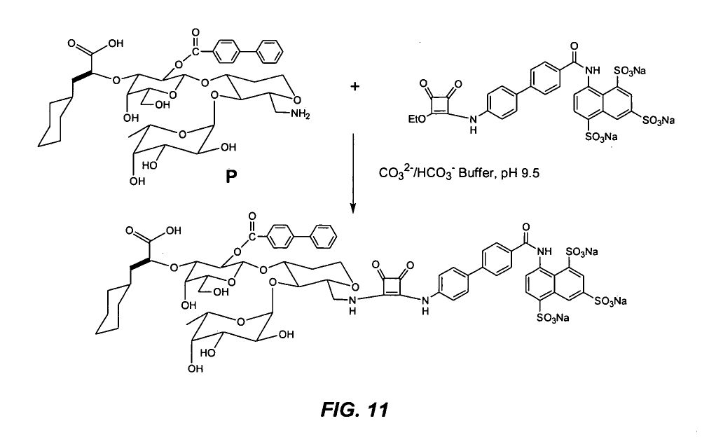

### Synthesis of Glycomimetic-BASA (FIG. 11)

Condensation Between BASA and Diethyl Squarate:

The BASA of Example 1 (10 mg) is reacted with diethyl squarate (5 mg) in phosphate buffer at pH 7 and then purified by preparative hplc to give the adduct A.

Synthesis of Glycomimetic P:

Glycomimetic P is synthesized as described in Example 9.

Condensation Between Glycomimetic P and Intermediate A:

To a solution of intermediate A (15 mg) in carbonate/bicarbonate buffer (pH 9.5, 1.5 ml) is added Glycomimetic P (10 mg) and the reaction mixture is stirred at room temperature for 16 h. The reaction mixture is then applied to column of sephadex G-25 and the column is eluted with 5 mM ammonium bicarbonate solution. The fractions that correspond to the product are collected and lyophilized to yield Glycomimetic-BASA conjugate (11 mg).

### Example 12

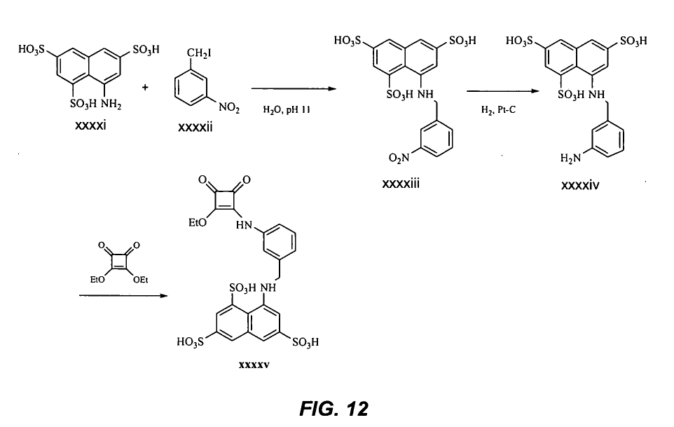

### Synthesis of a BASA and BASA-Squarate (FIG. 12)

Synthesis of BASA:

3-nitro-benzyl iodide is added to an aqueous solution (pH 11) of commercially available, 8-aminonaphthalene-1,3,5-trisulfonic acid (xxxxxi) with stirring at room temperature. pH of the solution is adjusted to 1 and after vaporation of the solvent, the product xxxxiii is precipitated out from ethanol.

Platinum catalyzed hydrogenation of compound xxxxiii affords BASA compound xxxxiv in 96% yield.

Synthesis of BASA-Squarate:

To a solution of compound xxxxiv in phosphate buffer (pH 7.1) is added commercially available diethyl squarate and the reaction mixture is stirred for 4 h at RT. It is then purified by reverse phase hplc to afford BASA-squarate compound xxxxv.

### Example 13

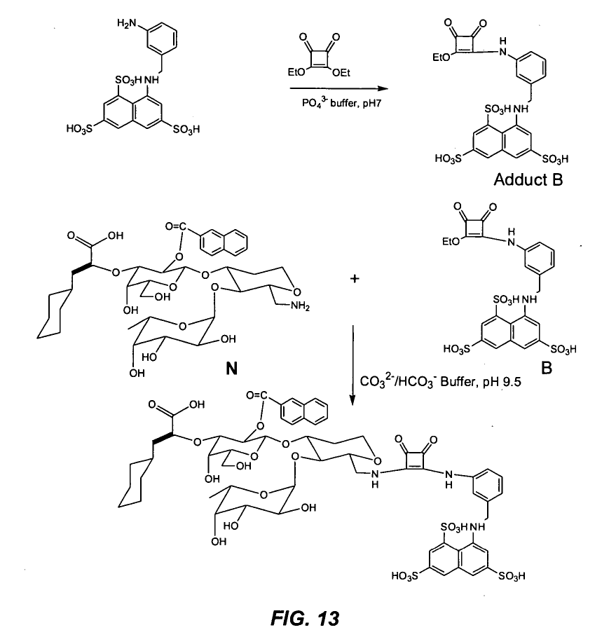

### Synthesis of Glycomimetic-BASA (FIG. 13)

Condensation Between BASA and Diethyl Squarate:

The BASA of Example 12 (10 mg) is reacted with diethyl squarate (5 mg) in phosphate buffer at pH 7 and then purified by preparative hplc to give the adduct B.

Synthesis of Glycomimetic N:

Glycomimetic N is synthesized as described in Example 8.

Condensation Between Glycomimetic N and Intermediate B:

To a solution of intermediate B (15 mg) in carbonate/bicarbonate buffer (pH 9.5, 1.5 ml) is added Glycomimetic N (10 mg) and the reaction mixture is stirred at room temperature for 16 h. The reaction mixture is then applied to column of sephadex G-25 and the column eluted with 5 mM ammonium bicarbonate solution. The fractions that correspond to the product are collected and lyophilized to yield Glycomimetic-BASA conjugate (14 mg).

### Example 14

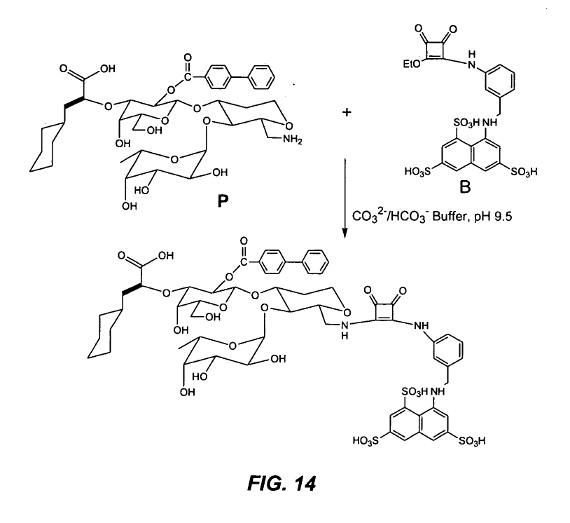

### Synthesis of Glycomimetic-BASA (FIG. 14)

Condensation Between BASA and Diethyl Squarate:

The BASA of Example 12 (10 mg) is reacted with diethyl squarate (5 mg) in phosphate buffer at pH 7 and then purified by preparative hplc to give the adduct B.

Synthesis of Glycomimetic P:

Glycomimetic P is synthesized as described in Example 9.

Condensation Between Glycomimetic P and Intermediate B:

To a solution of intermediate B (15 mg) in carbonate/bicarbonate buffer (pH 9.5, 1.5 ml) is added Glycomimetic P (10 mg) and the reaction mixture is stirred at room temperature for 16 h. The reaction mixture is then applied to column of sephadex G-25 and the column is eluted with 5 mM ammonium bicarbonate solution. The fractions that correspond to the product are collected and lyophilized to yield Glycomimetic-BASA conjugate (15 mg).

### Example 15

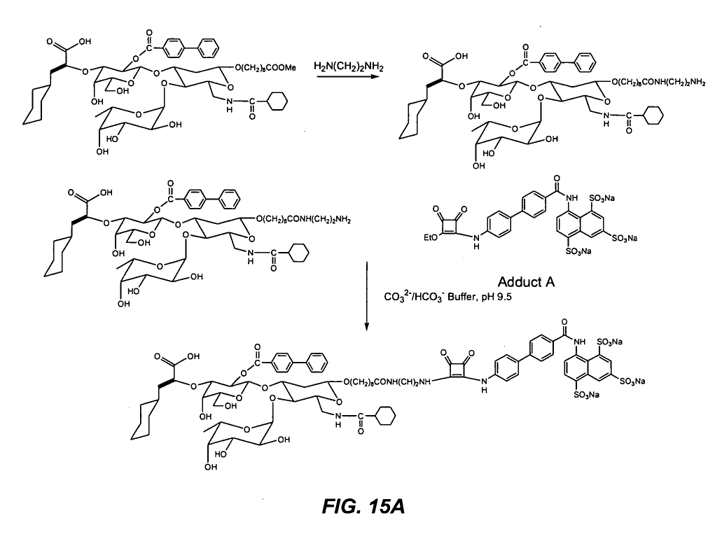

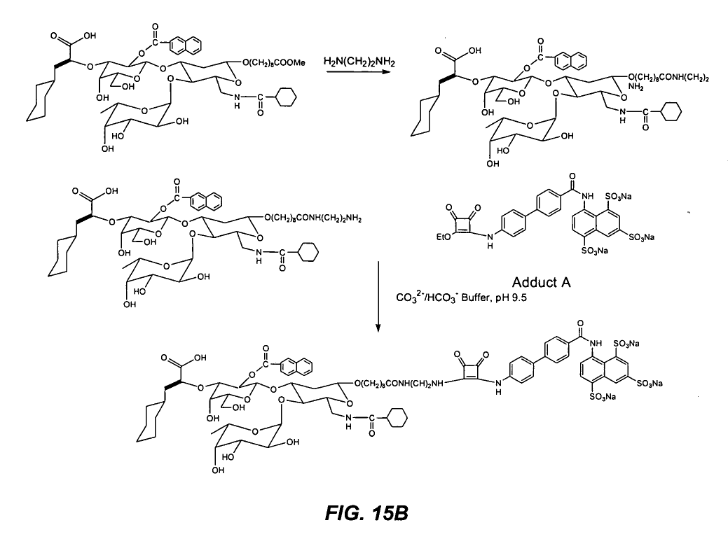

### Synthesis of Glycomimetic-BASA (FIGS. 15A and 15B)

Synthesis of BASA-Squarate (Intermediate A):

This reaction is performed as described in Example 10.

Synthesis of Compound 29:

Compound 28 of Example 7 is treated with excess of ethylenediamine at 70 for 5 h and then solvent is evaporated off. The crude product is purified by sephadex G-25 column to give compound 29.

Conjugation Between Compound 29 and BASA-Squarate:

Compound 29 is added to a solution of BASA-squarate in carbonate/bicarbonate buffer at pH 9.5 and the reaction mixture is stirred at room temperature for 16 h. It is then purified by sephadex G-25 column to give Glycomimetic-BASA conjugate.

### Example 16

### Synthesis of Glycomimetic-BASA

Synthesis of BASA-Squarate (Intermediate B):

This reaction is performed as described in Example 13.

Conjugation Between Compound 29 and BASA-Squarate:

Compound 29 of Example 15 is added to a solution of BASA-squarate in carbonate/bicarbonate buffer at pH 9.5 and the reaction mixture is stirred at room temperature for 16 h. It is then purified by sephadex G-25 column to give Glycomimetic-BASA conjugate.

### Example 17

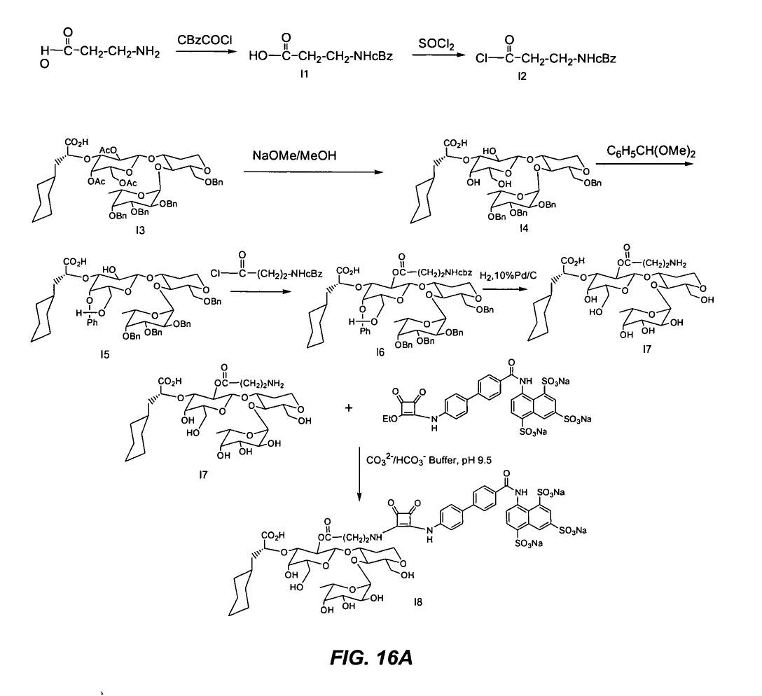

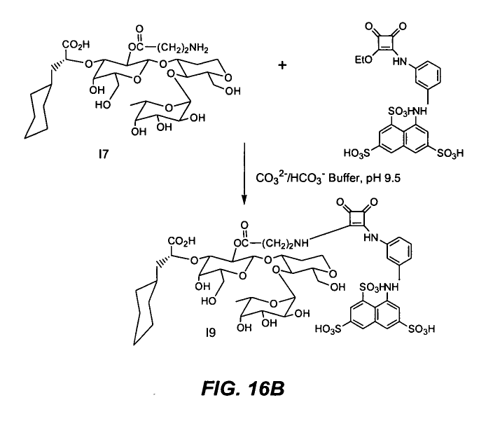

### Synthesis of Glycomimetic-BASA (FIGS. 16A and 16B)

Synthesis of I1 and I2:

To an aquous solution of commercially available b-alanine is added conc. HCl. The solution is diluted with ethanol and is added dropwise a solution benzylcarbonochloride in dimethoxyethane with stirring. The stirring is continued for 24 h. After usual work up the reaction mixture is purified hplc to give intermediate I1.

To solution of I1 in DMF is added thionyl chloride and the reaction mixture is stirred at RT for 1 h. Solvent is evaporated off and is purified by hplc to give I2.

Synthesis of Compound I7:

Synthesis of starting material I3: This compound is synthesized in a manner similar to that described in Example 7 and depicted in FIG. 7A.

Synthesis of Intermediate I4:

Compound I3 is treated with 0.1M NaOMe in MeOH 4 h at room temperature and then is neutralized with IR-120(H+) resin to give compound I2.

Synthesis of Intermediate I5:

To a solution of I4 in acetonitrile is added benzaldehyde dimethyl acetal and p-toluenesulfonic acid. The reaction mixture is stirred at room temperature for 4 h and neutralized with triethylamine. Solvent is evaporated off and the crude product is purified by column chromatography to give I5.

Synthesis of Intermediate I6:

To a solution of I5 in pyridine is added a 2,6-dimethylamino pyridine followed by the addition of I2. The reaction mixture is stirred at room temperature for 16 h and solvent is evaporated off. The crude reaction mixture is purified by column chromatography to give intermediate I6.

Hydrogenation of Intermediate I6:

To a solution of intermediate I6 in dioxan is added 10% Pd—C and the reaction mixture is shaken vigorously at room temperature for 24 h. Catalyst is filtered off through a celite bed and the supernatant concentrated to dryness to give compound I7.

Synthesis of Glycomimetic-BASA (FIG. 16A)

Conjugation between I7 and BASA-squarate adduct: To a solution of BASA-squarate adduct (from Example 10) in carbonate/bicarbonate buffer (pH 9.5) is added compound I7 and the reaction mixture is stirred at room temperature for 16 h. The reaction mixture is purified by sephadex G-25 to give Glycomimetic-BASA compound I8.

Synthesis of Glycomimetic-BASA (FIG. 16B)

Conjugation between I7 and BASA-squarate adduct: To a solution of BASA-squarate adduct (from Example 12) is added in carbonate/bicarbonate buffer (pH 9.5) is added compound I7 and the reaction mixture is stirred at room temperature for 16 h. The reaction mixture is purified by sephadex G-25 to give Glycomimetic-BASA compound 19.

### Example 18

### Assay for E-Selectin Antagonist Activity

Wells of a microtiter plate (plate 1) are coated with E-selectin/hlg chimera (GlycoTech Corp., Rockville, Md.) by incubation for 2 hr at 37° C. After washing the plate 5 times with 50 mM Tris HCl, 150 mM NaCl, 2 mM CaCl₂, pH 7.4 (Tris-Ca), 100 μl of 1% BSA in Tris-Ca/Stabilcoat (SurModics, Eden Prairie, Minn.) (1:1, v/v) are added to each well to block non-specific binding. Test compounds are serially diluted in a second low-binding, round bottomed plate (plate 2) in Tris-Ca (60 μl/well). Preformed conjugates of SLea-PAA-biotin (GlycoTech Corp., Rockville, Md.) mixed with Streptavidin-HRP (Sigma, St. Louis, Mo.) are added to each well of plate 2 (60 μl/well of 1 μg/ml). Plate 1 is washed several times with Tris-Ca and 100 μl/well are transferred from plate 2 to plate 1. After incubation at room temperature for exactly 2 hours the plate is washed and 100 μl/well of TMB reagent (KPL labs, Gaithersburg, Md.) is added to each well. After incubation for 3 minutes at room temperature, the reaction is stopped by adding 100 μl/well of 1M H₃PO₄ and the absorbance of light at 450 nm is determined by a microtiter plate reader.

### Example 19

### Assay for P-Selectin Antagonist Activity

The neoglycoprotein, sialylLe^{a}-HSA (Isosep AB, Sweden) is coated onto wells of a microtiter plate (plate 1) and the wells are then blocked by the addition of 2% bovine serum albumin (BSA) diluted in Dulbecco's phosphate-buffered saline (DPBS). In a second microtiter plate (plate 2), test antagonists are serially diluted in 1% BSA in DPBS. After blocking, plate 1 is washed and the contents of plate 2 are transferred to plate 1. Pselectin/hlg recombinant chimeric protein (GlycoTech Corp., Rockville, Md.) is further added to each well in plate 1 and the binding process is allowed to incubate for 2 hours at room temperature. Plate 1 is then washed with DPBS and peroxidase-labelled goat anti-human Ig(γ) (KPL Labs, Gaithersburg, Md.) at 1 μg/ml is added to each well. After incubation at room temperature for 1 hour, the plate is washed with DBPS and then TMB substrate (KPL Labs) is added to each well. After 5 minutes, the reaction is stopped by the addition of 1M H₃PO₄. Absorbance of light at 450 nm is then determined using a microtiter plate reader.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A compound or physiologically acceptable salt thereof, having the formula: 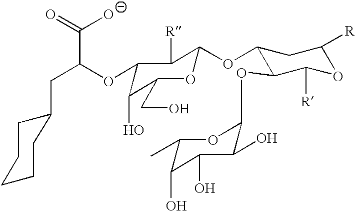
wherein:
R=H or a benzyl amino sulfonic acid;
R′=a benzyl amino sulfonic acid, 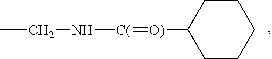 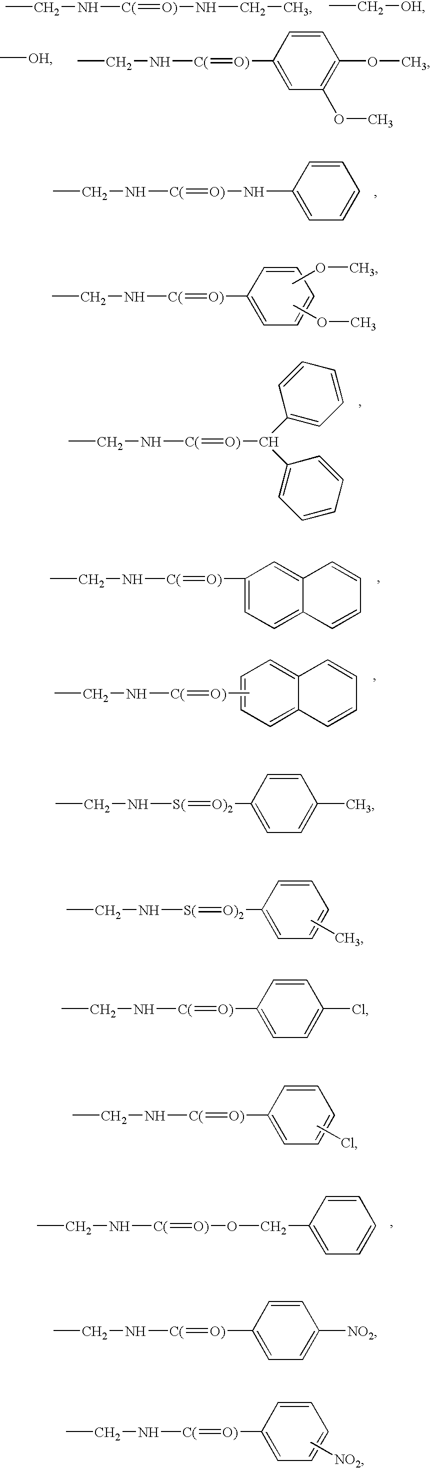 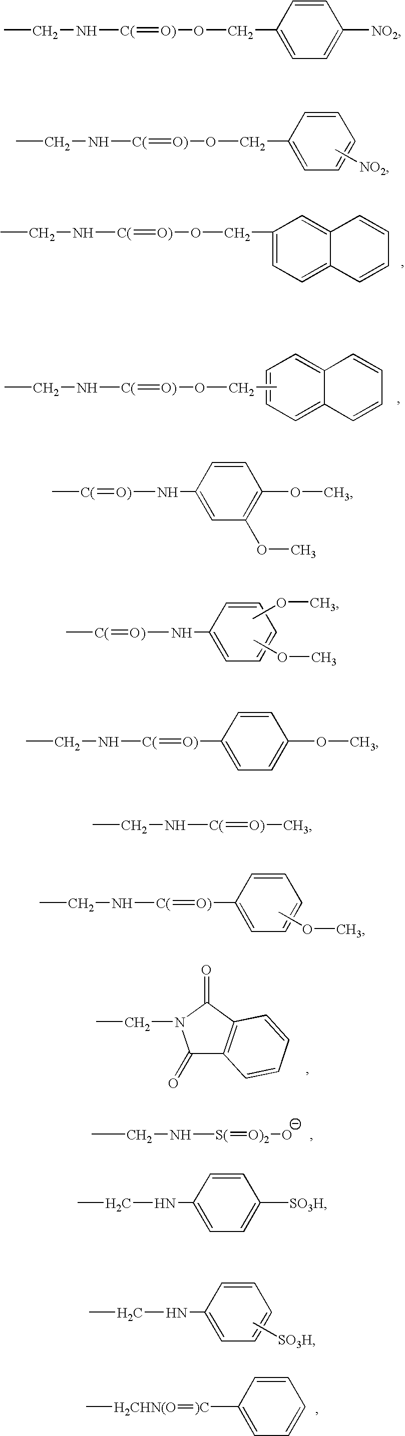 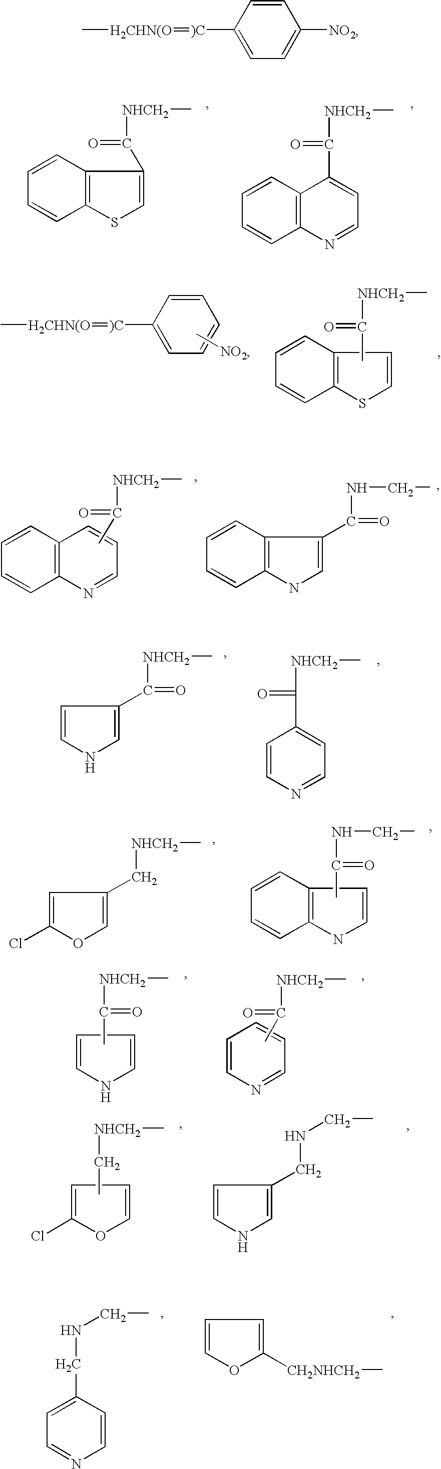 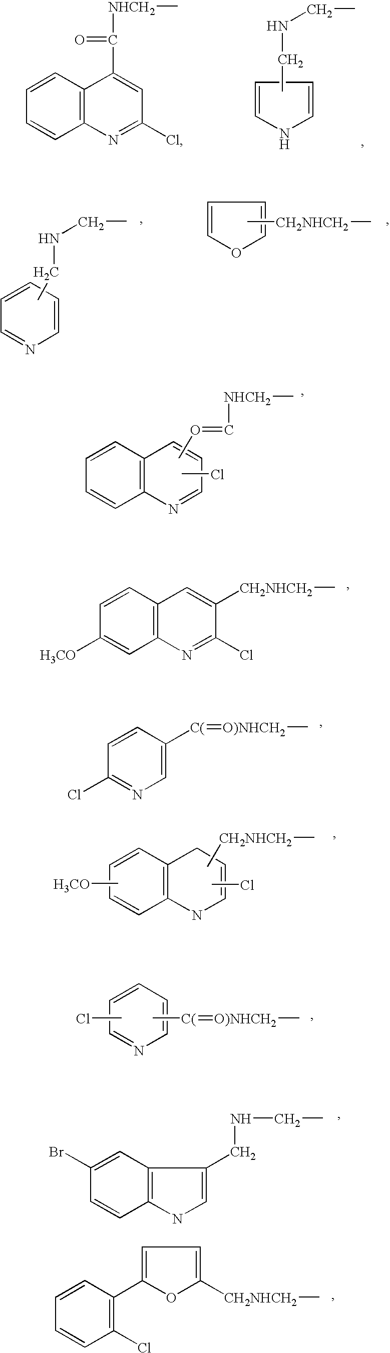 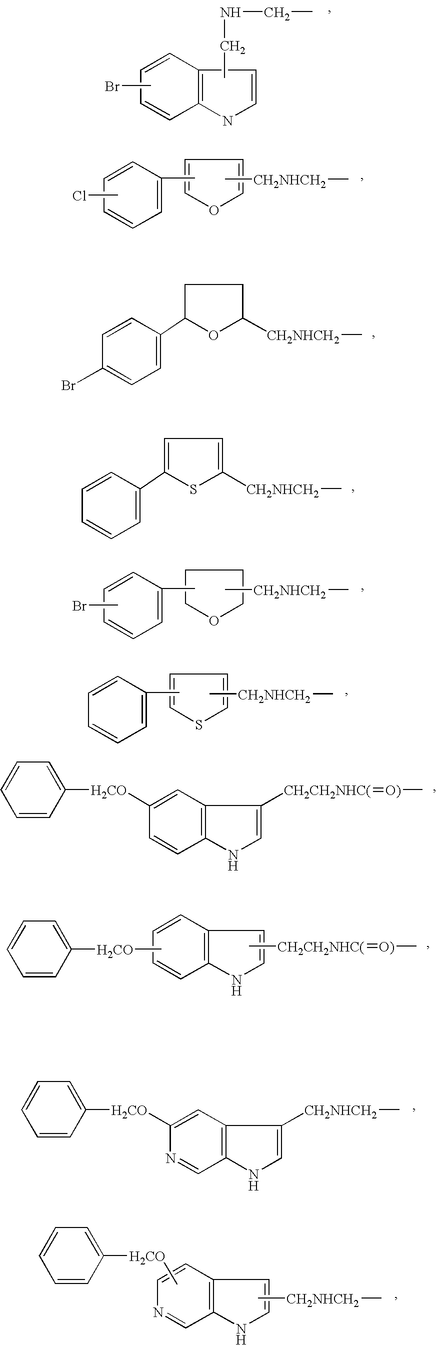 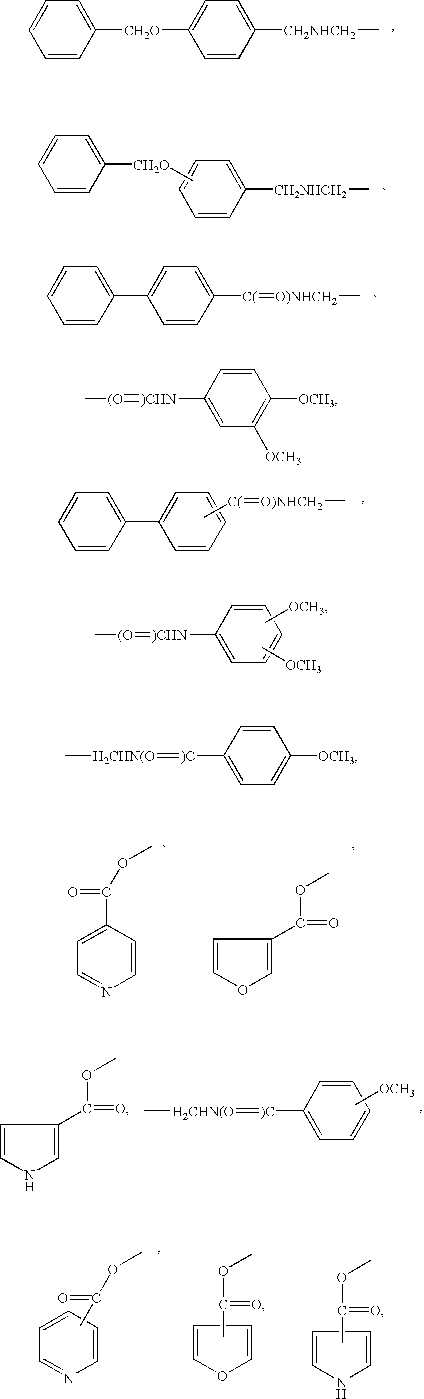 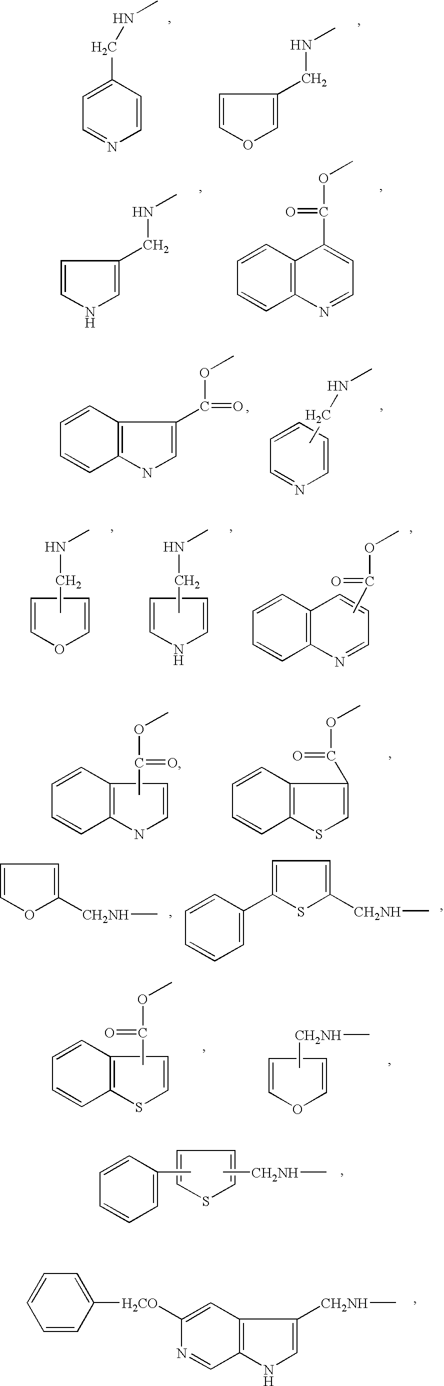 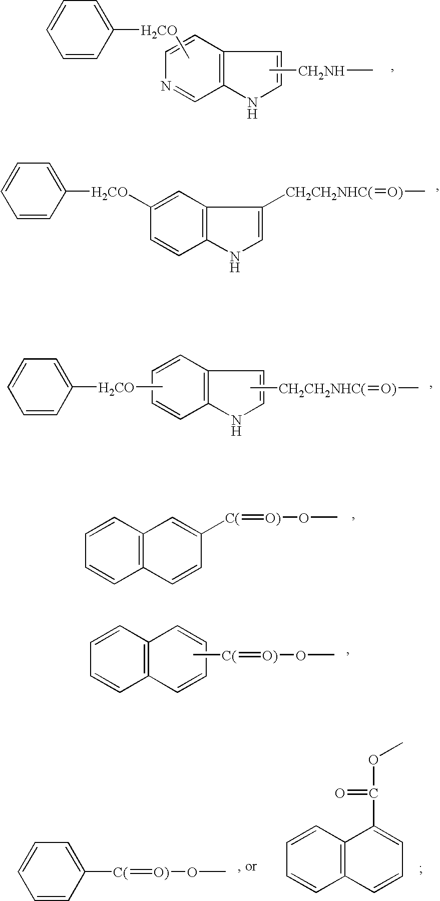
R″=a benzyl amino sulfonic acid, —OH, —OC(═O)—NH—CH₂—CH₃, 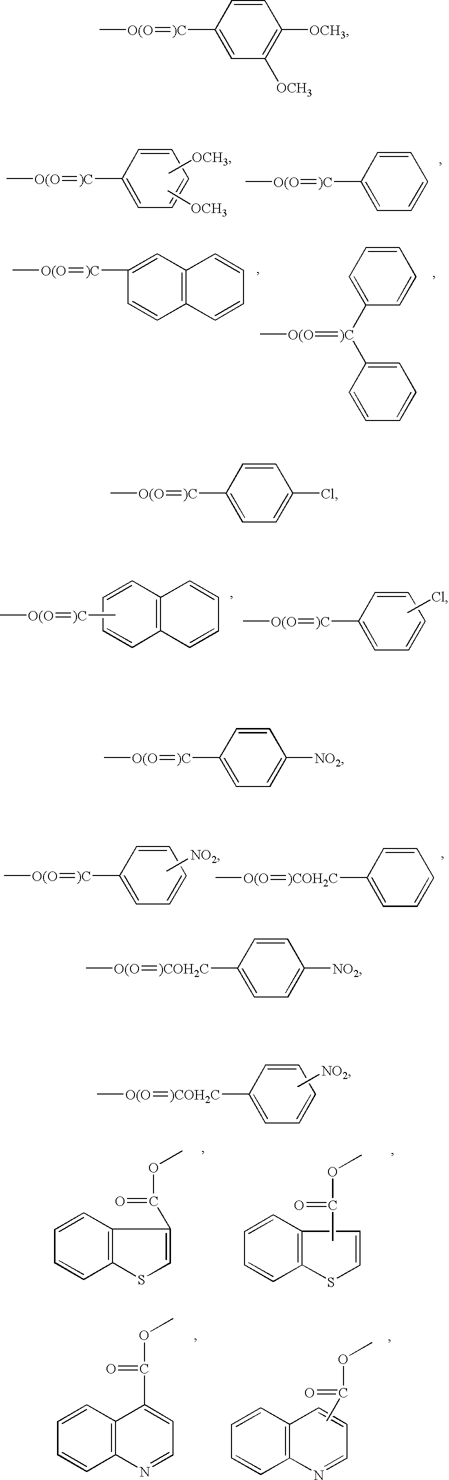 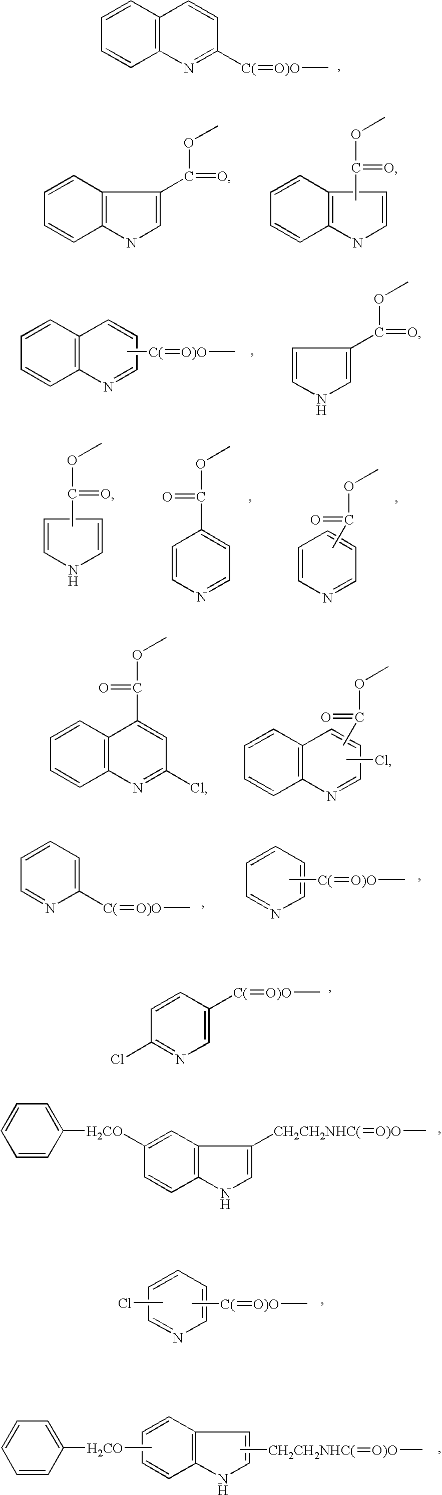 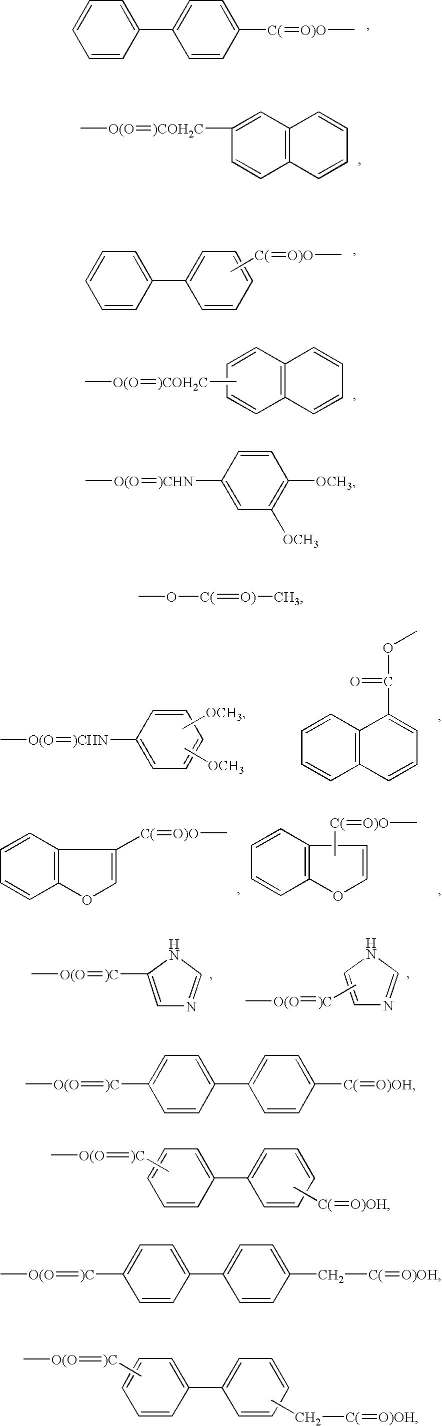 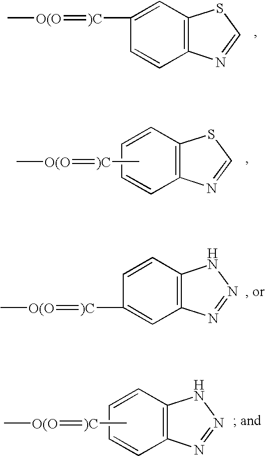
wherein the compound possesses a benzyl amino sulfonic acid at R, R′ or R″ but not at more than one of R, R′ and R″.

2. A compound or salt thereof according to claim 1 wherein R is a benzyl amino sulfonic acid.

3. A compound or salt thereof according to claim 2 wherein R″ is —OH.

4. A compound or salt thereof according to claim 2 wherein R′ is not —OH.

5. A compound or salt thereof according to claim 1 wherein R is H and R′ is a benzyl amino sulfonic acid.

6. A compound or salt thereof according to claim 5 wherein R″ is not —OH.

7. A compound or salt thereof according to claim 1 wherein R is H and R″ is a benzyl amino sulfonic acid.

8. A compound or salt thereof according to claim 7 wherein R′ is not —OH.

9. A composition comprising a compound or salt thereof according to any one of claims 1-8 in combination with a pharmaceutically acceptable carrier or diluent.

10. A compound or physiologically acceptable salt thereof comprising a compound or salt thereof according to any one of claims 1-8 further comprising a diagnostic or therapeutic agent.

11. A composition comprising a compound or salt thereof according to claim 10 in combination with a pharmaceutically acceptable carrier or diluent.

12. A method for modulating a selectin-mediated function, comprising contacting a cell expressing a selectin with a compound or salt thereof according to any one of claims 1-8 in an amount effective to modulate the selectin's function.

13. A method for modulating a selectin-mediated function, comprising contacting a cell expressing a selectin with a composition according to claim 9 in an amount effective to modulate the selectin's function.

14. A method of treating a patient, comprising administering to the patient who is in need of having inhibited the development of a condition associated with an excessive selectin-mediated function, a compound or salt thereof according to any one of claims 1-8 in an amount effective to inhibit the development of such a condition.

15. A method of treating a patient, comprising administering to the patient who is in need of having inhibited the development of a condition associated with an excessive selectin-mediated function, a composition according to claim 9 in an amount effective to inhibit the development of such a condition.

16. A method of inhibiting rejection of transplanted tissue, comprising administering to a patient who is the recipient of a transplanted tissue, a compound or salt thereof according to any one of claims 1-8 in an amount effective to inhibit rejection of the transplanted tissue.

17. A method of inhibiting rejection of transplanted tissue, comprising administering to a patient who is the recipient of a transplanted tissue, a composition according to claim 9 in an amount effective to inhibit rejection of the transplanted tissue.

18. A method of targeting an agent to a selectin-expressing cell, comprising contacting a cell expressing a selectin with a compound or salt thereof according to claim 10 in an amount effective to target a diagnostic or therapeutic agent to the cell.

19. A method of targeting an agent to a selectin-expressing cell, comprising contacting a cell expressing a selectin with a composition according to claim 11 in an amount effective to target a diagnostic or therapeutic agent to the cell.
